(19) United States
(12) Patent Application Publication
(10) Pub. No.: US 2003/0003088 A1
(43) Pub. Date: Jan. 2, 2003

(54) Human pancreatic pluripotential stem cell line

(75) Inventors: Ming Sound Tsao, Toronto (CA); James E. Trosko, Okemos, MI (US); Burra V. Madhukar, Okemos, MI (US); Lawrence K. Olson, East Lansing, MI (US); Loretta VanCamp, East Lansing, MI (US)

Correspondence Address: MCLEOD & MOYNE, 2190 COMMONS PARKWAY, OKEMOS, MI, 48864

(73) Assignee: Board of Trustees operating Michigan State University, East Lansing, MI

(21) Appl. No.: 10/135,801

(22) Filed: Apr. 30, 2002

Related U.S. Application Data
(60) Provisional application No. 60/288,473, filed on May 3, 2001.

(51) Int. Cl. A61K 48/00; C12N 5/08; C12N 15/86

(52) U.S. Cl. 424/93.21; 435/366; 435/456

(57) ABSTRACT

A human pancreatic ductal epithelial cell line immortalized with the human papilloma virus E6 and E7 genes which has stem cell-like characteristics and which can be induced to differentiate into ductal-like cells and beta-like cells that produce insulin. The immortal cells or derivative thereof are useful for treating insulin-dependent diabetes and in assays for determining the ability of a chemical to induce pancreatic stem cell differentiation or malignancy.

### CROSS-REFERENCE TO RELATED APPLICATION

Not Applicable.

### STATEMENT REGARDING FEDERALLY SPONSORED RESEARCH OR DEVELOPMENT

Not Applicable.

### REFERENCE TO A “COMPACT DISC APPENDIX”

Not Applicable.

### BACKGROUND OF THE INVENTION

(1) Field of the Invention

The present invention relates to a human pancreatic ductal epithelial cell line immortalized with the human papilloma virus E6 and E7 genes which has stem cell-like characteristics and which can be induced to differentiate into ductal-like cells and beta-like cells that produce insulin. The immortal cells or derivative thereof are useful for treating insulin-dependent diabetes and in assays for determining the ability of a chemical to induce pancreatic stem cell differentiation or malignancy.

(2) Description of Related Art

Understanding the complex, multistage, multi-mechanism process of carcinogenesis, including that of human pancreatic cancer, requires characterizing the genotype and phenotype of those cells that can give rise to the cancers. Pancreatic cancer represents one of the leading causes of cancer deaths in many developed countries (Wingo et al., CA-Cancer J. Clin. 45: 8-30 (1995)). Following the isolation and immortalization of normal human pancreatic ductal epithelial cells transformed with human papilloma virus (HPV) type 16 E6 and E7 genes (Furukawa et al., Amer. J. Pathol. 148: 1763-1770 (1996)), the partial characterization of several critical genes in primary normal human pancreatic duct epithelial cells, in the pre- and post-immortalized derivative clones of the cells, and in several human pancreatic carcinoma cell lines has been reported (Liu et al., Am. J. Pathol. 153: 263-269 (1998); Ouyang et al., Am. J. Pathol. 157: 1623-1621 (2000)).

One of the oldest theories on the origin of cancers is that cancer is a disease of differentiation (Markert, Cancer Res. 28: 1908-1914 (1968); Pierce, Am. J. Pathol. 77: 103-118 (1974)); a stem cell disease (Till, J. Cell Physiol. Suppl. 1: 3-11 (1982)), or oncology as partially blocked ontogeny (Potter, Br. J. Cancer 38: 1-23 (1978)). In addition, normal cells are characterized as being under growth control and having the ability to terminally differentiate and to be mortal. In solid tissues, it is believed that gap junctional intercellular communication (GJIC) is responsible, in large part, for contact inhibition or growth control (Loewenstein, Biochim. Biophys. Acta 560: 1-65 (1979)), for control of differentiation (Warner, Semin. Cell Biol. 3: 81-91 (1992)), and apoptosis (Trosko and Goodman, Mol. Carcinog. 11: 8-12 (1994); Wilson et al., Exp. Cell Res. 254: 257-268 (2000)). Most normal cells in solid tissues express one of a number of evolutionarily-conserved genes coding for gap junction proteins: the connexins (Bruzzone et al., Eur. J. Biochem. 44: 947-951 (1996)). On the other hand, cancer cells are characterized as having lost growth control, having the inability to terminally differentiate, and being immortal. One of the unique characteristics of cancer cells is their inability to have functional homologous or heterologous GJIC, because of suppressed transcription of connexin genes, abnormal translation of connexin genes, translocation of connexin genes to other sites in the chromosome, abnormal assembly of connexins into connexons in the membrane, or abnormal functioning of gap junctions (Trosko and Ruch, Frontiers in Biosciences 3: 208-236 (1998)).

Recent studies suggest that at least some stem cells do not express connexin genes or have functional GJIC. The totipotent stem cell or fertilized egg does not have functional GJIC (Lee et al., Cell 51: 851-860 (1987)). The pluri-potent stem cells of the human kidney epithelium (Chang et al., Cancer res. 47: 1634-1645 (1987)), the human breast epithelium (Kao et al., Carcinogenesis 16: 531-538 (1995)), the corneal epithelium (Matic et al., Differentiation 61: 251-260 (1997)), or the human neuronal-glial (Dowling-Warriner and Trosko, Neurosciences 95: 859-868 (2000)) do not have functional GJIC. The human kidney, human breast, and human neural-glial epithelial pluri-potent stem cells can be induced to express connexins, which increases functional GJIC and differentiation (Kao et al., Carcinogenesis 16: 531-538 (1995); Dowling-Warriner and Trosko, Neurosciences 95: 859-868 (2000)). Because cancer cells are similar to stem cells and do not have functional GJIC, normal growth control, normal differentiation, or apoptosis, stem cells may be the target cells for carcinogenesis.

One of the major hypotheses concerning the carcinogenic process applicable to solid tissues has been the idea that GJIC is first reversibly down-regulated by endogenous (growth factors or hormones) or exogenous (chemical tumor promoters) agents during the tumor promotion phase and then stably down-regulated by alterations in activated oncogenes, e.g., ras, scr, neu, or the loss of tumor suppressor genes during the progression phase of carcinogenesis (Trosko et al., In: New Frontiers in Cancer Causation. O. H. Iverson (ed.), Taylor and Francis Publishers, Washington, D.C., pp. 181-197 (1993)). Evidence consistent with this hypothesis includes the observations that normal, but non-pluripotent cells, have functional GJIC; that most tumor cells have either dysfunctional homologous or heterologous GJIC; that most, if not all, tumor promoting chemicals reversibly inhibit GJIC; that growth factors can reversibly down-regulate GJIC; that activated oncogenes can stably down-regulate GJIC; that several tumor suppressor genes can up-regulate GJIC; that transfection of tumor cells with several connexin genes can restore GJIC and normalized growth control; that transfection of an anti-sense connexin gene can induce a “tumorigenic-like” phenotype in normal cells (Trosko and Ruch, Frontiers in Biosciences 3: 208-236 (1998); Trosko et al., In: New Frontiers in Cancer Causation. O. H. Iverson (ed.), Taylor and Francis Publishers, Washington, D.C., pp. 181-197 (1993)); and that a connexin32 knock-out mouse is highly predisposed to spontaneous and chemically-induced liver cancers (Temme et al., Curr. Biol. 7: 713-716 (1997)).

It has previously been shown that the cultured human pancreatic duct epithelial cells and immortalized but non-tumorigenic cell lines derived thereof resemble cells of the normal human pancreatic duct epithelium in vivo. However, it was unknown whether these cells can communicate via gap junctions or undergo differentiation under various growth conditions. If such cells could undergo differentiation, then they could be used in assays to determine the effect of particular chemicals on differentiation of pancreatic cells and the ability of particular chemicals to induce malignancy or prevent malignancy. Furthermore, provided the cells can be induced to differentiate, then the cells could be differentiated into insulin-producing cells which would be useful in therapies to treat insulin-dependent diabetes mellitus (IDDM, Type 1 diabetes).

Current investigations into treatments for IDDM has focused on transplantable devices that contain insulin-producing pancreatic cells. Theses devices or artificial pancreata have been designed to maintain the pancreatic cells in a physiological environment that protects the cells from destruction by the host's immune system. Various embodiments of transplantable devices or artificial pancreata are disclosed in U.S. Pat. No. 5,425,764 to Fournier et al., U.S. Pat. No. 5,702,444 to Struthers et al., U.S. Pat. No. 5,741,334 to Mullon et al., U.S. Pat. No. 5,885,613 to Antanavich et al., U.S. Pat. No. 5,855,616 to Fournier et al., U.S. Pat. No. 5,980,889 to Butler et al., U.S. Pat. No. 5,997,900 to Wang et al., U.S. Pat. No. 6,023,009 to Stegemann et al., and U.S. Pat. No. 6,165,225 to Antanavich et al. All of the aforementioned rely on providing islet of Langerhans cells which are differentiated pancreatic cells that produce insulin. Islet cells are not immortal cells and can be maintained in culture for a limited period of time. Therefore, the aforementioned devices must be replenished with islet cells from time to time. Furthermore, because islet cells need to be isolated from pancreatic tissue, the above devices must rely on organ donors for a supply of the islet cells. Therefore, considerable research effort has been devoted to means for maintaining the islet cells, making artificial insulin-producing cells, immortalizing islet cells, or isolating stem cells that can differentiate into islet cells.

U.S. Pat. No. 5,681,587 to Halberstadt et al. discloses a method for increasing the number of adult pancreatic islet cells available for transplantation. U.S. Pat. No. 5,993,799 to Newgard discloses a method for genetically engineering an anterior pituitary cell line immortalized with Rous sarcoma virus with an insulin gene, a glucokinase gene, and a glucose transporter gene to provide artificial beta cells that can secrete insulin in response to glucose. U.S. Pat. No. 4,332,893 to Rosenberg discloses a method for producing an insulin-producing conditionally-transformed beta cell line. The cell line is transformed with a Rous sarcoma virus with a temperature sensitive lesion in the viral transforming or sarc gene that enables the cell line to be propagated in vitro at the permissive temperature, which is a temperature different than the in vivo temperature. U.S. Pat. Nos. 5,795,790, 5,840,576, 5,843,431, 5,853,717, 5,858,747, and 5,935,849, all to Schinstine et al., disclose methods for controlling proliferation, distribution, differentiation of immortalized cells in artificial organs. U.S. Pat. No. 6,001,647 to Peck et al. discloses a method for isolating pancreatic stem cells, propagating the cells in vitro, and inducing the cells in vitro to differentiate into islet structures which can be used for implantation into a mammal for in vivo therapy of diabetes.

Thus, there remains a need to have a well-characterized human pancreatic pluri-potent stem cell line to be used for the molecular understanding of the genes needed for the development and differentiation of these cells into insulin-producing cells, to determine how they can form three-dimensional “organoids,” to use as a means to screen agents that induce or inhibit differentiation of insulin-producing cells and tissues, to study pancreatic carcinogenesis, and to use in treatments to replace insulin in diabetic patients.

### SUMMARY OF THE INVENTION

The present invention provides a human pancreatic ductal epithelial cell line immortalized with the human papilloma virus E6 and E7 genes which has stem cell-like characteristics and which can be induced to differentiate into ductal-like cells and beta-like cells that produce insulin. The immortal cells are useful for treating insulin-dependent diabetes and in assays for determining the ability of a chemical to induce pancreatic stem cell differentiation or malignancy.

Therefore, the present invention provides a human pancreatic ductal cell line immortalized with human papilloma virus genes E6 and E7 and which is capable of producing insulin, wherein the cells are gap junctional intercellular communication competent and are capable of expressing connexin43 gap junction protein upon induction by agents stimulating the production of cyclic AMP.

In a preferred embodiment, the present invention provides an immortalized human pancreatic ductal cell line capable of producing insulin and expressing connexin43 gap junction protein derived by differentiation from normal human pancreatic duct epithelium gap junctional intracellular communication incompetent cells transfected with human papilloma virus genes E6 and E7 and available from Michigan State University, East Lansing, Mich. or the Department of Laboratory Medicine and Pathobiology, University Health Network, Toronto, Ontario, Canada. In particular embodiments, the cells are maintained in a medium comprising a three-dimensional matrix, which produces the connexin43 protein.

Thus, the present invention provides a pluripotent human pancreatic ductal cell line immortalized with human papilloma virus genes E6 and E7 and which is capable of producing insulin, wherein the cells are (i) contact inhibited in complete keratinocyte serum-free medium containing growth factors, hormones and bovine pituitary extract (KSFM), (ii) capable of forming tubular/ductal structures in a medium comprising a three-dimensional matrix, (iii) gap junction intercellular communication competent in keratinocyte basal medium (KBM), and (iv) capable of expressing connexin 32 and 43 genes in KBM comprising c-AMP elevating agents. In particular, the cell line HPDE6c7 deposited as ATCC ______. The cell line is preferably maintained as the pluripotent stem cell line in KSFM and is preferably maintained as a differentiated cell line in a medium selected from the group consisting of KBM, KBM with c-AMP elevating agents, and medium comprising a three-dimensional matrix. Preferably, the c-AMP elevating agents are selected from the group consisting of 3-isobutyl-1-methylxanthine, forskolin, and mixture thereof.

The present invention also provides a method for screening a chemical agent for determining an affect on cells which comprises: (a) providing a human pancreatic ductal cell line immortalized with human papilloma virus genes E6 and E7 and which is capable of producing insulin, wherein the cells are gap junctional communication competent and are capable of expressing connexin43 gap junction protein upon induction by agents stimulating the production of cyclic AMP; and (b) exposing the cell line to the chemical agent to screen the effect of the chemical agent on the cell line. In a preferred embodiment, the immortalized human pancreatic ductal cell line is derived by differentiation from normal human pancreatic duct epithelium gap junctional intracellular communication incompetent cells transfected with human papilloma virus genes E6 and E7 and available from Michigan State University, East Lansing, Mich. or the Department of Laboratory Medicine and Pathobiology, University Health Network, Toronto, Ontario, Canada. In one embodiment, the cells are maintained in a medium comprising a three-dimensional matrix, which produces the connexin43 protein.

The present invention further provides a method for differentiating cells which comprises: (a) providing normal human pancreatic duct epithelium cells containing human papilloma virus genes E6 and E7, wherein the cells are gap junctional intracellular connection incompetent and are incapable of producing insulin and connexin43; and (b) maintaining the cells of step (a) with a cyclic AMP elevating agent in basal medium, without hormones and growth factors, to produce the differentiated cells which are gap junctional intracellular connection competent and which produce connexin43 gap junction protein. Preferably, the cells are an immortalized human pancreatic ductal cell line derived by differentiation from normal human pancreatic duct epithelium gap junctional intracellular communication incompetent cells transfected with human papilloma virus genes E6 and E7 and available from Michigan State University, East Lansing, Mich. Preferably, the cells are maintained in a medium comprising a three-dimensional matrix which enables production of the connexin43 protein. In a preferred embodiment, the chemical agent is tested on the cell line for an ability to cause the cell line to become tumorigenic or is tested on the cell line for an ability to affect the production of insulin.

In a preferred embodiment, the present invention provides a method for determining the ability of a chemical agent to affect differentiation of insulin-producing cells or tissues, which comprises: (a) providing a pluripotent human pancreatic ductal cell line immortalized with human papilloma virus genes E6 and E7 and which is capable of producing insulin, wherein the cells are (i) contact inhibited in complete keratinocyte serum-free medium containing growth factors, hormones and bovine pituitary extract (KSFM), (ii) capable of forming tubular/ductal structures in a medium comprising a three-dimensional matrix, (iii) gap junction intercellular communication competent in keratinocyte basal medium (KBM) , and (iv) capable of expressing connexin 32 and 43 genes in KBM comprising c-AMP elevating agents; (b) exposing the cell line to the chemical agent in complete medium or basal medium with or without c-AMP elevating agents; and (c) determining the effect of the chemical agent on differentiation. Preferably, the cell line is HPDE6c7 deposited as ATCC ______ and the c-AMP elevating agents are selected from the group consisting of 3-isobutyl-1-methylxanthine, forskolin, and mixture thereof.

The present invention further provides a method for determining the ability of a chemical agent to affect production of insulin, which comprises: (a) providing a pluripotent human pancreatic ductal cell line immortalized with human papilloma virus genes E6 and E7 and which is capable of producing insulin, wherein the cells are (i) contact inhibited in complete keratinocyte serum-free medium containing growth factors, hormones and bovine pituitary extract (KSFM), (ii) capable of forming tubular/ductal structures in a medium comprising a three-dimensional matrix, (iii) gap junction intercellular communication competent in keratinocyte basal medium (KBM), and (iv) capable of expressing connexin 32 and 43 genes in KBM comprising c-AMP elevating agents; (b) exposing the cell line to the chemical agent in complete medium or basal medium with or without c-AMP elevating agents; and (c) determining the effect of the chemical agent on production of insulin. Preferably, the cell line is HPDE6c7 deposited as ATCC ______ and the c-AMP elevating agents are selected from the group consisting of 3-isobutyl-1-methylxanthine, forskolin, and mixture thereof.

The present invention further provides a method for determining the ability of a chemical agent to affect differentiation of insulin-producing cells or tissues, which comprises (a) providing a pluripotent human pancreatic ductal cell line immortalized with human papilloma virus genes E6 and E7 and which is capable of producing insulin, wherein the cells are (i) contact inhibited in complete keratinocyte serum-free medium containing growth factors, hormones and bovine pituitary extract (KSFM), (ii) capable of forming tubular/ductal structures in a medium comprising a three-dimensional matrix, (iii) gap junction intercellular communication competent in keratinocyte basal medium (KBM), and (iv) capable of expressing connexin 32 and 43 genes in KBM comprising c-AMP elevating agents; (b) exposing the cell line to the chemical agent in complete medium or basal medium with or without c-AMP elevating agents; and (c) determining the effect of the chemical agent on differentiation. The method claim 21, wherein the human papilloma virus genes E6 and E7 are provided by a plasmid or a recombinant virus.

Preferably, the method wherein the cell line is HPDE6c7 deposited as ATCC ______. In a further embodiment of the method, the c-AMP elevating agents are selected from the group consisting of 3-isobutyl-1-methylxanthine, forskolin, and mixture thereof.

The present invention further provides a method for treating type-I diabetes in a mammal comprising: (a) providing a therapeutically effective amount of a human pancreatic ductal cell line immortalized with human papilloma virus genes E6 and E7 and which is capable of producing insulin, wherein the cells are gap junctional intercellular communication competent and are capable of expressing connexin43 gap junction protein upon induction by agents stimulating the production of cyclic AMP, positioned in a means for producing an artificial pancreas; and (b) implanting the artificial pancreas in the mammal wherein the artificial pancreas produces insulin. Preferably, the immortalized human pancreatic ductal cell line is derived by differentiation from normal human pancreatic duct epithelium gap junctional intracellular communication incompetent cells transfected with human papilloma virus genes E6 and E7 and available from Michigan State University, East Lansing, Mich. In a preferred embodiment, the artificial pancreas comprises the immortalized cell line positioned within a selectively permeable device which is connected to the vasculature of the mammal.

The present invention further provides a human pancreatic ductal epithelial cell line wherein the cells of the cell line are immortalized with an agent selected from the group consisting of human papilloma virus (HPV) genes E6 and E7, SV40 T antigen, Rous sarcoma virus, one or more oncogenes selected from the group consisting of ras, scr, and neu, and a chemical mutagen selected from the group consisting of N-methyl-N-nitro-N-nitrosoguanidine (MNNG), methyl methane sulfonate (MMS), nitrosourea (NMU), dimethylbenz[a]anthracine (DBMA), 4-nitroquinoline-N-oxide (NQO), and nickel (II) and which is capable of producing insulin, wherein the cells are gap junctional intercellular communication competent and are capable of expressing connexin43 gap junction protein upon induction by agents stimulating the production of cyclic AMP.

Finally, the present invention provides a method for making an immortalized human pancreatic ductal epithelial cell line which is capable of producing insulin, wherein the cells are gap junctional intercellular communication competent and are capable of expressing connexin43 gap junction protein upon induction by agents stimulating the production of cyclic AMP, comprising (a) isolating ductal tissue from human pancreatic tissue; (b) incubating the ductal tissue in a cell culture to form a monolayer of cells growing from the ductal tissue; (c) treating the monolayer of cells with an agent selected from the group consisting of human papilloma virus (HPV) genes E6 and E7, SV40 T antigen, Rous sarcoma virus, one or more oncogenes selected from the group consisting of ras, scr, and neu, and a chemical mutagen selected from the group consisting of N-methyl-N-nitro-N-nitrosoguanidine (MNNG), methyl methane sulfonate (MMS), nitrosourea (NMU), dimethylbenz[a]anthracine (DBMA), 4-nitroquinoline-N-oxide (NQO), and nickel (II) for a time sufficient to immortalize the cells; and (d) growing the immortalize cells for a time sufficient to allow the cells that are not immortalized to die to produce the immortalized cell line, wherein the immortalized cell line is capable of producing insulin, and wherein the immortalized cells of the cell line are gap junctional intercellular communication competent and are capable of expressing connexin43 gap junction protein upon induction by agents stimulating the production of cyclic AMP.

### Objects

Therefore, it is an object of the present invention to provide an immortal pancreatic stem cell line that can be used in transplants to treat insulin-dependent diabetes, in assays to determine the ability of a chemical to induce stem cell differentiation, and in assays to determine the potential for a chemical to induce a stem cell to become malignant.

These and other objects of the present invention will become increasingly apparent with reference to the following drawings and preferred embodiments.

### DESCRIPTION OF THE DRAWINGS

FIG. 1A is a phase-contrast microphotograph that shows the morphology of the cells of the present invention (HPDE6c7) on plastic cell culture dishes. The HPDE6c7 cells were seeded to culture dishes or multi-well dishes with growth factor-free medium as described in Example 2 for 2 to 3 days. Note the epithelial morphology of the cells. Magnification was ×200.

FIG. 1B is a phase-contrast microphotograph that shows the morphology of the HPDE6c7 cells grown in MATRIGEL. The HPDE6c7 cells were seeded to culture dishes or multi-well dishes with growth factor-free MATRIGEL as described in Example 2 for 2 to 3 days. Note the ductal organization with budding structures of the cells. Magnification was ×200.

FIG. 2 is a phase-contrast microphotograph that shows gap junctional intercellular communication (GJIC) in HPDE6c7 cells cultured in complete growth medium (A or B) or in basal medium with IBMX and forskolin for 2 days (C and D). GJIC was assessed using Lucifer yellow dye transfer as described in Example 3. Note the absence of dye transfer from the primary dye loaded cells into the contacting neighboring cells in B. A significant increase in dye-coupled cells after treatment with IBMX/forskolin for 48 hours is shown in D. A and C are phase-contrast pictures. Magnification was ×200.

FIG. 3 shows a quantitative analysis of GJIC in HPDE6c7 cells incubated under particular growth conditions. HPDE6c7 cells were cultured in complete growth medium (KSFM) or basal medium without growth factors or hormones (KBM) in the presence or absence of c-AMP elevating agents. GJIC was assessed by the Lucifer yellow dye transfer method and quantified using an image analysis program. Each bar represents the mean of three different assays ±SEM.

FIG. 4 shows a Western immunoblot of connexin43 protein expression in HPDE6c7 cells grown in KBM with or without c-AMP. Cells were treated with c-AMP elevating agents for different times and the level of connexin43 proteins was determined by immunoblotting as described in Example 4. Note the increase in the amount of connexin43 protein as well as the phosphorylated form of connexin43 in c-AMP treated cells.

FIG. 5 shows the results of an RT-PCR assay for detecting expression of RNA encoding connexin32 and 43 in the HPDE6c7 cells grown in KBM containing c-AMP elevating agents for 48 hours. HPDE6c7 cultures in KBM were treated for 48 hours with IBMX and forskolin. Total RNA was extracted and used for RT-PCR as described in Example 5.

FIG. 6 shows the results of an RT-PCR assay for detecting expression of RNA encoding connexin45 in HPDE6c7 cells under particular culture conditions. HPDE6c7 cells were incubated in KBM (lanes 2 and 7), KGM containing 10 mM nicotinamide (lanes 3 and 8), KGM containing 100 μM c-AMP (lanes 4 and 9), or KGM containing 10 mM nicotinamide and 100 μM c-AMP (lanes 5 and 10) for 4 days. Afterwards, total RNA was isolated as in Example 5 and connexin45 (lanes 2 through 5) and beta-actin (lanes 7 through 10) gene expression was measured by RT-PCR as in Example 5. Lanes 1, 6, and 11 are molecular weight markers.

FIG. 7 is a phase-contrast microphotograph that shows that particular cells in a monolayer of HPDE6c7 cells grown on plastic cell culture dishes in KBM accumulate zinc. The cells were stained with dithizone, which stains cells that accumulate zinc.

FIG. 8A is a phase-contrast microphotograph showing a monolayer of HPDE6c7 cells infected with adenovirus vector (AdINSGFP), which expresses Green Fluorescence Protein (GFP) under the regulation of the insulin promoter, incubated in KBM for three days.

FIG. 8B is a fluorescence microphotograph showing that particular cells in the monolayer of AdINSGFP-infected HPDE6c7 cells incubated in KBM for three days express the GFP.

FIG. 9A is a phase-contrast microphotograph showing a monolayer of AdINSGFP-infected HPDE6c7 cells incubated in KBM containing 10 mM nicotinamide for three days.

FIG. 9B is a fluorescence microphotograph showing that a substantial number of cells in the monolayer of AdINSGFP-infected HPDE6c7 cells incubated in KBM containing 10 mM nicotinamide for three days express GFP.

FIG. 10 shows that the HPDE6c7 cells express RNA encoding insulin. Lane 1 is the molecular weight markers. Lanes 2 through 7 show the RT-PCR product using primers for Pdx-1, lanes 8 through 13 show the RT-PCR product using primers for insulin, and lanes 14 through 19 show the RT-PCR product using primers for beta-actin. Lanes 2, 8, and 14 show the RT-PCR product for HPDE6c7 cells incubated in RPMI-1640 medium; lanes 3, 9, and 15 show the RT-PCR product for HPDE6c7 cells incubated in complete KSF medium; lanes 4, 10, and 16 show the RT-PCR product for HPDE6c7 cells incubated in KBM medium; lanes 5, 11, and 17 show the RT-PCR product for HPDE6c7 cells incubated in KBM medium containing 10 mM nicotinamide; lanes 6, 12, and 18 show the RT-PCR product for HPDE6c7 cells incubated in KBM media containing betacellulin; and, lanes 7, 13, and 19 show the RT-PCR product for HPDE6c7 cells incubated in KBM medium containing 10 mM nicotinamide and 3 mM betacellulin.

FIG. 11 is a schematic representation of an embodiment of an artificial pancreas suitable for use with the HPDE6c7 cells or derivative thereof of the present invention.

FIG. 12 shows the results of an assay in which HPDE6c7 cells were treated with polycyclic aromatic hydrocarbons (PAH) under forskolin induction of GJIC over a 72 hour time span. PAH was added at every medium change. The “% change over control at −30 min” is the per cent GJIC in the cells at time of measurement compared to GJIC in the cells at seeding.

FIG. 13 shows the results of an assay in which HPDE6c7 cells were treated with PAH under forskolin effect over a 72 hour time span. PAH was added at time −30 minutes only. The “% change over control at −30 min” is the per cent GJIC in the cells at time of measurement compared to GJIC in the cells at seeding.

### DETAILED DESCRIPTION OF THE INVENTION

All patents, patent applications, government publications, government regulations, and literature references cited in this specification are hereby incorporated herein by reference in their entirety. In case of conflict, the present description, including definitions, will control.

The present invention provides immortalized human pancreatic stem cells, which are pluripotent and can be induced to differentiate into ductal epithelium cells or into insulin-producing cells. The remarkable ability of the cells to differentiate into insulin-producing cells indicates that the cells are useful as a novel and unlimited source of human pancreatic beta cells. The beta cells derived from the immortalized pancreatic stem cells can be used for producing insulin in vitro but more importantly, the beta cells so derived can be used for transplantation therapies for treating insulin-dependent diabetes (IDDM).

The immortal human pancreatic stem cells of the present invention are human pancreatic ductal epithelial (HPDE) cells immortalized with human papilloma virus (HPV) genes E6 and E7, SV40 T antigen, Rous sarcoma virus, oncogenes such as ras, scr, or neu, or a chemical mutagen such as N-methyl-N-nitro-N-nitrosoguanidine (MNNG), methyl methane sulfonate (MMS), nitrosourea (NMU), dimethylbenz[a]anthracine (DBMA), 4-nitroquinoline-N-oxide (NQO), or nickel (II). In a preferred embodiment, the present invention provides human pancreatic ductal epithelial clone 7 cells (HPDE-6-E6E7C7 which is hereinafter referred as HPDE6c7) and derivative thereof, which are a clonal population of HPDE cells derived from HPDE cells immortalized by transfecting the cells with an amphotrophic retrovirus containing human papilloma virus (HPV) 16 genes E6 and E7 (Furukawa et al. Amer. J. Path. 148: 1763-1770 (1996)). The HPDE6c7 cells are available from the Department of Pediatrics and Human Development, Michigan State University, East Lansing, Mich., USA or the Department of Laboratory Medicine and Pathobiology, University Health Network, Toronto, Ontario, Canada. The HPDE6c7 cells were deposited under the terms of the Budapest Treaty at the American Type Culture Collection, 10801 University Boulevard, Manassas, Va. 20110-2209 as ATCC ______.

The HPDE6c7 cells show anchorage-dependent growth in cell culture, and the HPDE6c7 cells are nontumorigenic when inoculated into Balb-C nude mice. The HPDE6c7 cells have retained pancreatic ductal epithelial cell characteristics which was reported by Lui, et al., Amer. J. Path. 153: 263-269 (1998). The HPDE6c7 cells also have a near normal diploid karyotype and express some of the phenotypes characteristic of normal pancreatic duct epithelial cells, including mRNA expression of the carbonic anhydrase II gene, MUC-1 gene, and the genes encoding cytokeratins 7, 8, 18, and 19. The HPDE6c7 cells have normal Ki-ras, p53, c-myc, and p16 (INK4A) genotypes, and cytogenetic studies demonstrate the loss of 3p, 10p12, and 13q14, the latter including the Rb1 gene. Consistent with the presence of the E6 gene product, wild-type p53 protein was detectable at very low levels. The lack of a functional p53 pathway can be shown by the inability of gamma-irradiation to up-regulate p53 and p21 waf1/cip1 proteins. Consistent with E7 protein expression, the p110/Rb protein was not detectable. (See Ouyang et al., Amer. J. Pathol. 157: 1623-1631 (2000)). The cells also express human cytokeratin 7, which indicate that the cells are derived from pancreatic duct epithelium, and the cells express vimentin and bcl-2, which are markers of pancreatic stem cells.

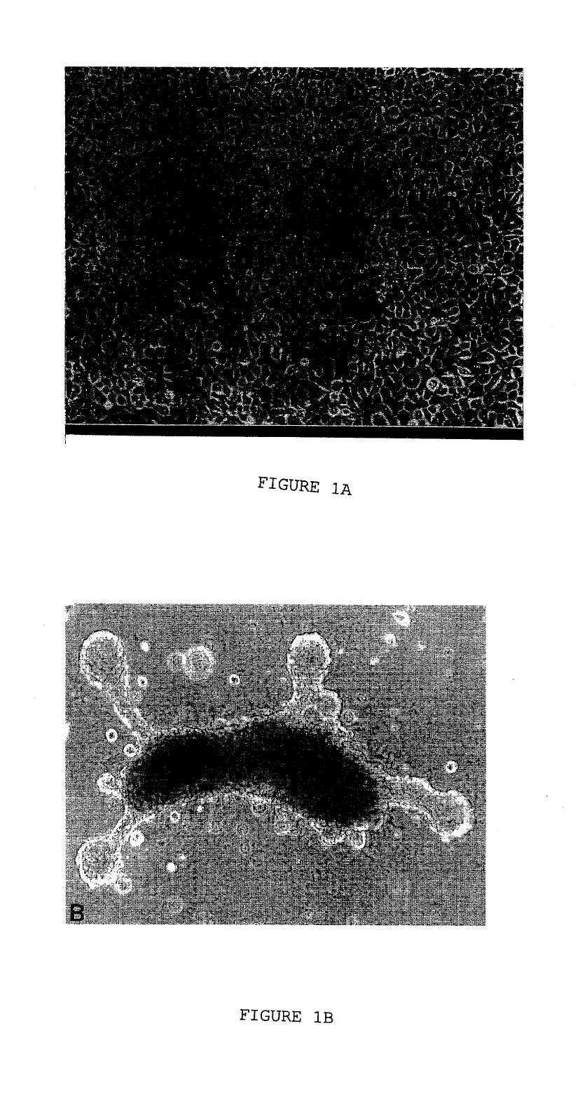

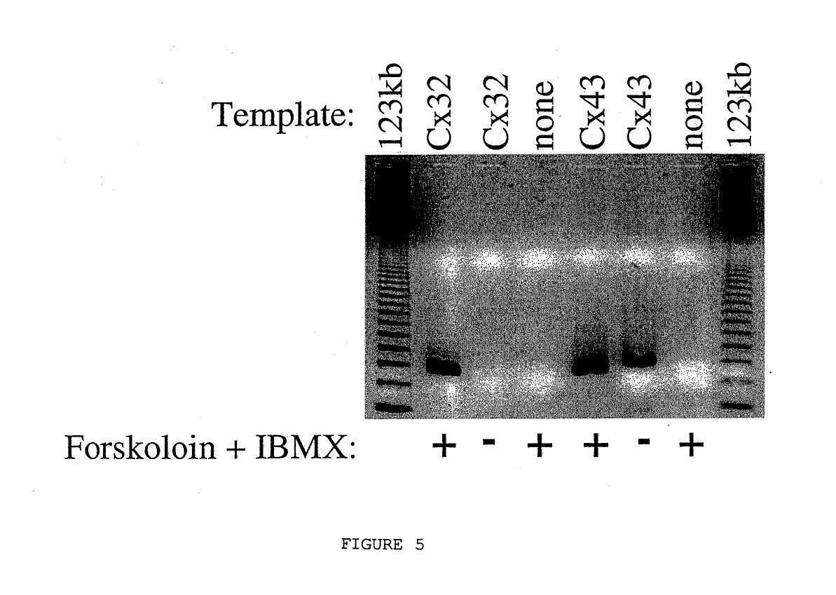

As shown herein, it has been discovered that the HPDE6c7 cells have retained pancreatic ductal epithelial stem cell characteristics and have the ability to differentiate into ductal-like structures and to express cystic fibrosis transmembrane conductance regulator (CFTR). The HPDE6c7 cells comprise stem cells because (1) the HPDE6c7 cells can divide symmetrically to expand its population (FIG. 1A) or divide asymmetrically to differentiate into pancreatic ductal cells (FIG. 1B), (2) the HPDE6c7 cells do not have functional gap junction intercellular communication (GJIC) and, therefore, are similar to other “toti-potent stem cells” or fertilized egg, and (3) the HPDE6c7 cells under particular growth conditions can be induced to form a three-dimensional “organoid” having several differentiated phenotypes such as functional GJIC, expressed connexin genes, expressed CFTR genes, all of which are not present in the cells when they are not induced. For example, culturing the HPDE6c7 cells in basal medium devoid of growth factors,. hormones, or pituitary extract induces the cells to significantly increase their GJIC as measured by the increase in connexin43 protein and connexin32 transcript, which are present in differentiated cells but not in stem cells (See FIG. 5). Likewise, culturing the HPDE6c7 cells in the presence of c-AMP elevating agents such as forskolin and 3-isobutyl-1-methylxanthine (IBMX) also induce the cells to increase their GJIC.

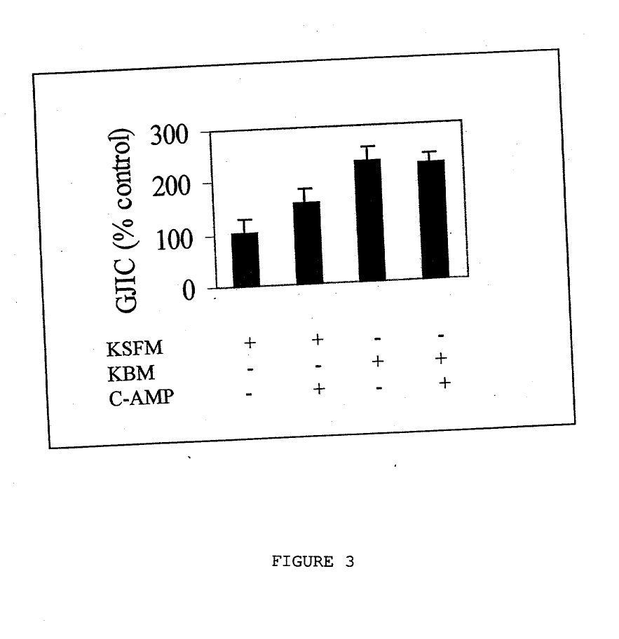

Thus, the absence of GJIC in these HPDE6c7 cells under normal cell culture growth conditions indicates that the cells have retained particular stem cell characteristics (See FIG. 3). For example, the HPDE6c7 cells do not have functional GJIC in a manner similar to the early embryo, human kidney, neural-glial, breast epithelial, and corneal epithelium stem cells. This important discovery implies that the HPDE6c7 cells have stem cell characteristics and that under proper growth conditions, the cells may be induced to differentiate into other pancreatic cell types such as insulin producing beta cells. As shown herein, the HPDE6c7 cells can be induced under particular cell culture conditions to become GJIC competent (See FIG. 3) and remarkably, under particular growth conditions, the HPDE6c7 cells can be induced to express insulin and secret measurable levels of insulin into cell culture medium. For example, when the HPDE6c7 cells are harvested from culture plates and re-plated to fresh culture plates, particular cells in the re-plated cell population express insulin for a period of time. Treating the cells with particular chemical agents will also induce the cells to turn on insulin production. For example, treating the HPDE6c7 cells with nicotinamide, cholera toxin, or sonic hedgehog will induce the HPDE6c7 cells to produce insulin. Bouwens et al. (J. Histochem. Cytochem. 44: 947-951 (1996)) has shown that the ductal epithelial cells of the pancreas are the embryonic origin of the hormone-producing cells of the pancreas. Consistent with the ductal origin of the hormone-producing cells of the pancreas, Bonner-Weir et al., Proc. Natl. Acad Sci. USA 97: 7999-8004 (2000), showed that ductal cells from adult human pancreata can be expanded in cell culture and then directed to differentiate into glucose responsive insulin-producing islet cells.

Therefore, because the HPDE6c7 cells of the present invention can be induced to form ductal-like structures (FIG. 1B), the ability of the HPDE6c7 cells to be induced to produce insulin is consistent with the cells being stem cells which can be induced to differentiate into insulin-producing cells. Agents that can increase beta cell differentiation or proliferation include, but are not limited to, nicotinamide, sodium butyrate, activin A, betacellulin, prolactin, placental lactogen, growth hormone (GH), insulin growth factors (IGF-1 and -2), hepatocyte growth factor (HGF), vascular endothelial growth factor (VEGF), basic fibroblast growth factor (bFGF), epithelial growth factor (EGF), transforming growth factor-alpha (TGF-α), and gastrin. Particular combinations of the aforementioned agents can be used to induce the HPDE6c7 cells to differentiate into insulin producing beta cells.

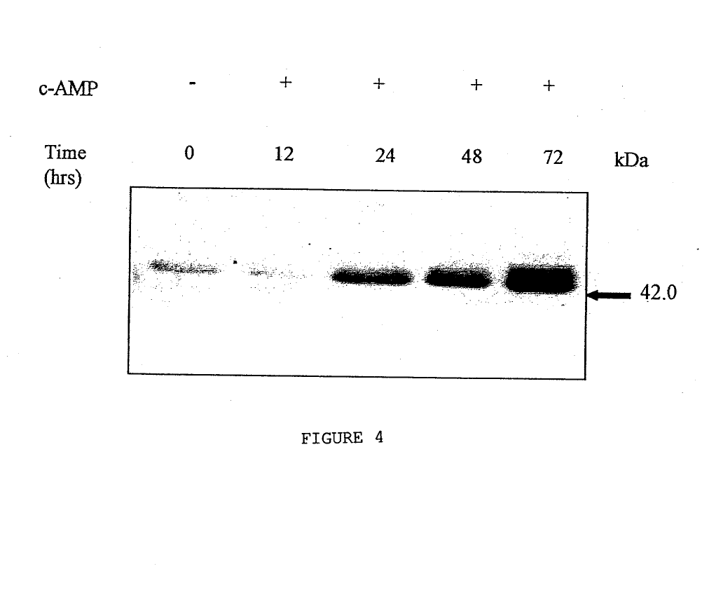

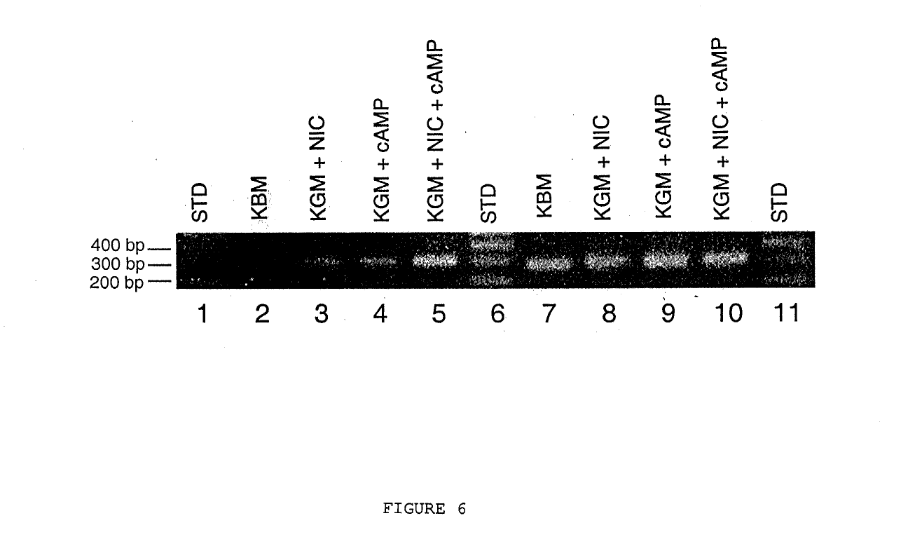

It has been shown that pancreatic beta cells express the following gap junction proteins: connexin36 and connexin45 (Serre-Beinier et al., Diabetes 49:727-734 (2000)); and, connexin43 (Bosco and Meda, In Gap Junctions. Werner, R. (Ed.). IOS Press, Amsterdam, Netherlands. pp. 153-157 (1998)). Similar to beta cells, the HPDE6c7 cells of the present invention express connexin43 protein and can be induced to increase expression of the connexin43 protein by incubating the cells with adenosine 3′5′-cyclic monophosphate (c-AMP) or c-AMP elevating chemicals such as forskolin (FIG. 4). The HPDE6c7 cells do not express the connexin32, 45, or 26 proteins and c-AMP was unable to induce their expression (FIG. 4). However, it was discovered that even though HPDE6c7 cells do not produce connexin32 or 45 protein, the cells did produce mRNA encoding connexin32 when incubated with forskolin and IBMX (FIG. 5) or mRNA encoding connexin45 under all culture conditions (FIG. 6). The HPDE6c7 cells express connexin36 when treated with forskolin for 72 hours but not when treated with c-AMP elevating agent KBM from 24 to 72 hours. The above results are consistent with the HPDE6c7 being capable of differentiating into beta cells under appropriate cell culture conditions and further suggests that when the HPDE6c7 cells are induced to produce insulin, they can function as a unit in the process of insulin secretion and release. More recently, it has been shown that in a transgenic cell line in which the gene encoding connexin43 had been functionally deleted by homologous recombination, embryonic development was similar to that in wild-type pancreas (Charollais et al., (Devel. Genet. 24: 13-26 (1999)). This implies that other connexins may have compensated for the loss of connexin43 in the development and function of the pancreas. Further examination indicated that the rat and mouse pancreas contained six connexin transcripts, including connexin45.

The ability of the HPDE6c7 cells of the present invention to differentiate into cells that express insulin under particular growth conditions indicates that the cells are useful for a therapeutic approach in the reversal of insulin-dependent diabetes mellitus (IDDM, Type 1 diabetes). Differentiation of pancreatic ductal stem cells into insulin-producing cells has the potential for therapeutic approaches to reversing IDDM Type 1 (Mashima et al., Diabetes 48: 304-309 (1999); Cornelius et al., Horm. Metab. Res. 29: 271-277 (1997); Rosenberg, Cell Transplant. 4: 371-383 (1995); Rosenberg and Vinik, Adv. Exp. Med. Biol. 321: 95-104 (1992); Korsgren et al., Surgery 113: 205-214 (1993)). Normally, patients with IDDM require daily injections of insulin to control hyperglycemia and proper utilization of blood glucose. Transplantation of insulin-producing human-derived cells can obviate the need for insulin injections to control hyperglycemia. The level of insulin currently expressed by the HPDE6c7 cells of the present invention is modest; however, the results shown herein clearly show that immortalization of human pancreatic ductal epithelium cells can sustain stem cells that are pluripotent and can differentiate into other types of cells of the pancreas. This is similar to the SV40 immortalized human neural-glia pluripotent stem cells which were induced to express GJIC by treatment with c-AMP elevating chemicals such as forskolin (Dowling-Warriner and Trosko, (2000)). The ability of the HPDE6c7 cells to differentiate into insulin-producing cells is further supported by the discovery that under conditions that induced differentiation, the cells expressed connexin43 protein and mRNA encoding connexin43, 32, and 45.

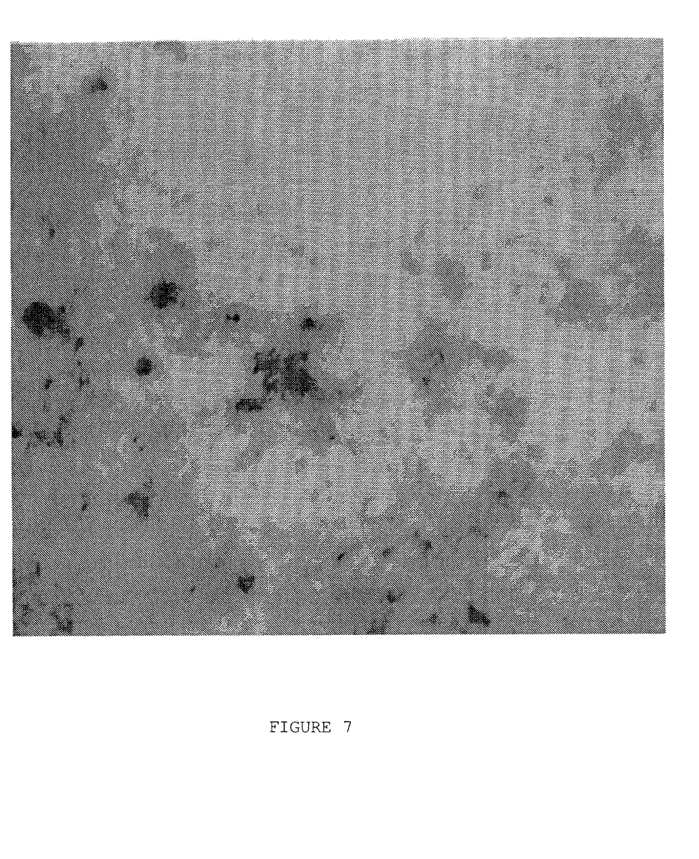

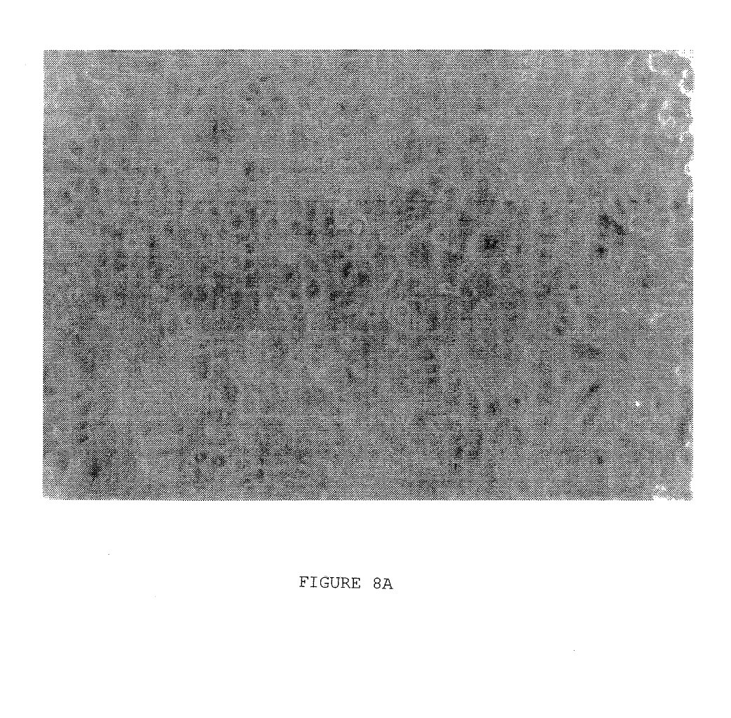

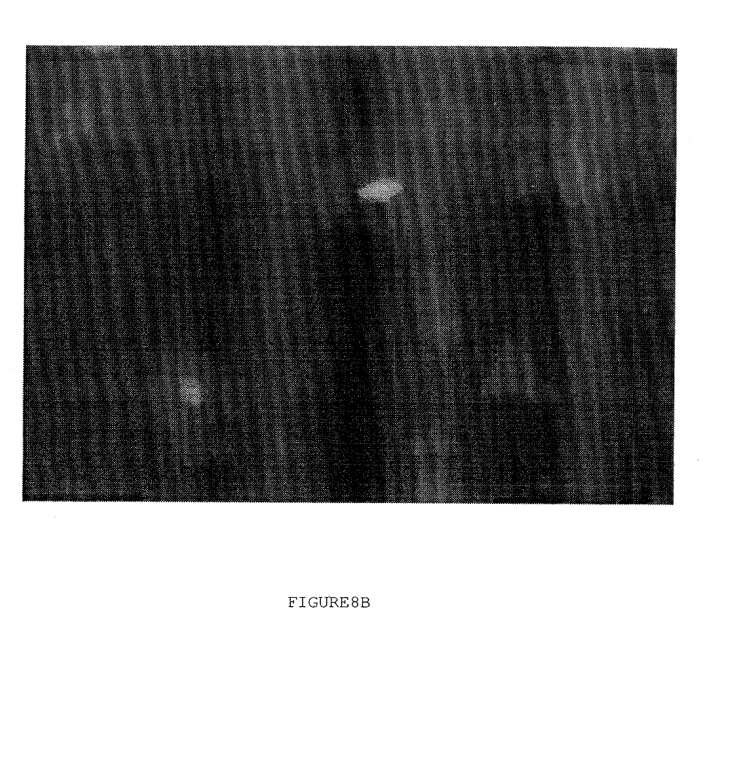

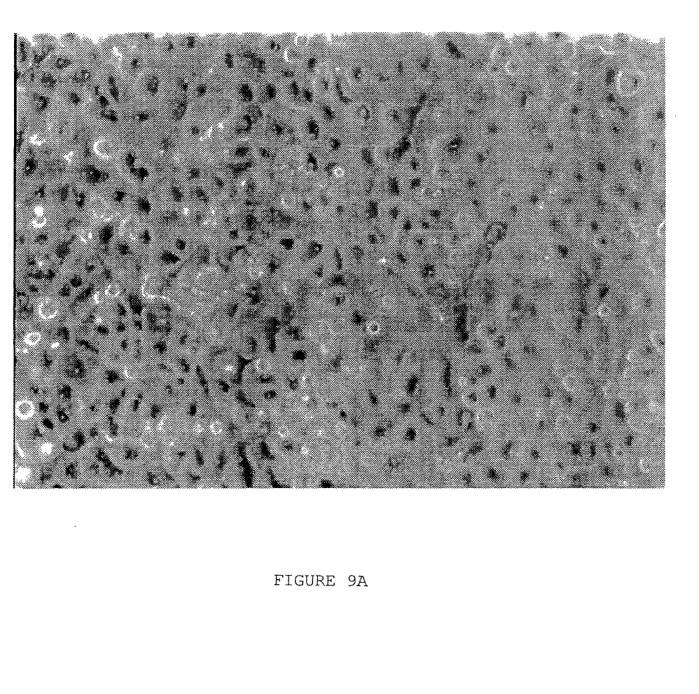

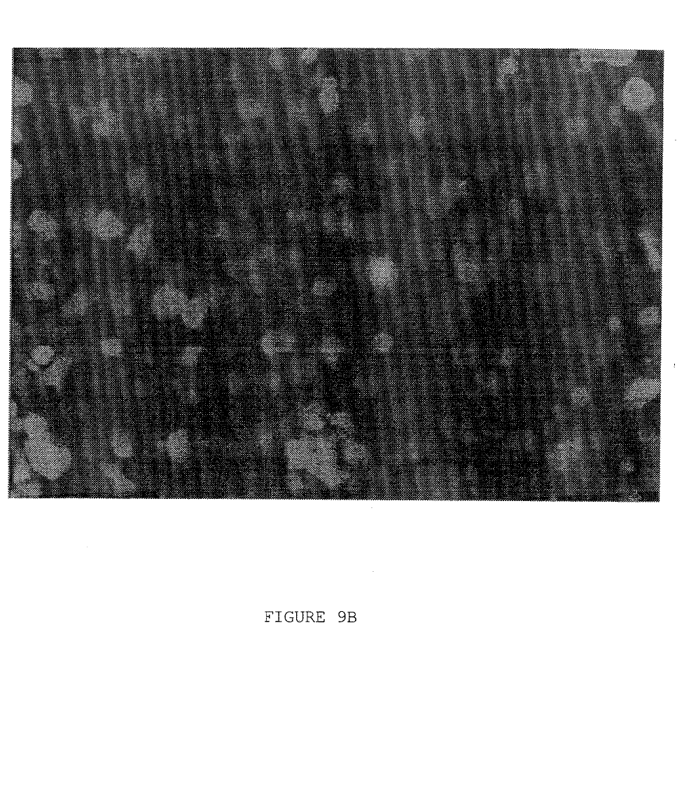

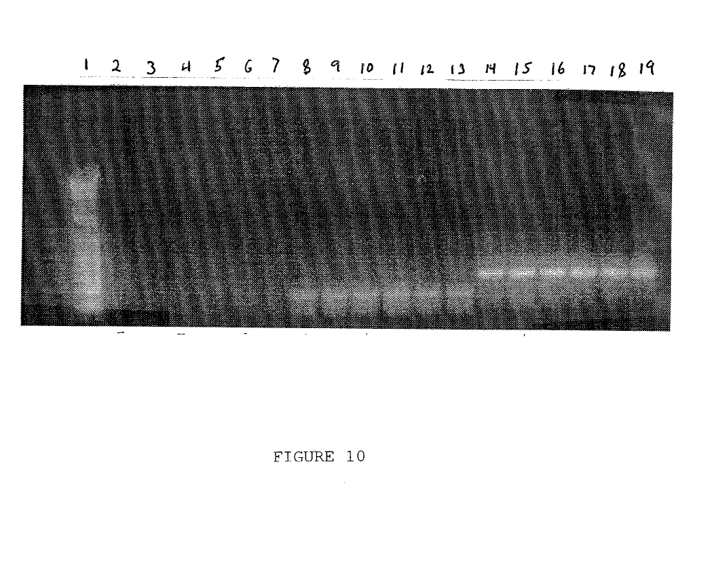

The HPDE6c7 cells have the capacity to produce insulin, which indicates that the cells can be induced to differentiate into insulin-producing cells. The following demonstrates the insulin-producing potential of the cells. First, the HPDE6c7 cells accumulate zinc and insulin is complexed with zinc. This is shown in FIG. 7, which shows that a monolayer of the cells grown on plastic dishes stain red with dithizone stain, an indicator for zinc. Second, the HPDE6c7 cell monolayer contains cells with the insulin gene promoter turned on. This is shown in FIG. 8B which shows that cells infected with an adenovirus that expresses Green Fluorescent Protein (FGP) under the regulation of the human insulin promoter fluoresce green. Thus, the HPDE6c7 cell monolayer contains cells that have active insulin transcription. Third, incubating the HPDE6c7 cell monolayer with nicotinamide increases the number of cells in the monolayer in which the insulin promoter is activated. This is shown in FIG. 9B which shows that growing a monolayer of HPDE6c7 cells infected with the adenovirus expressing the GFP under the regulation of the human insulin promoter in media containing nicotinamide markedly increased the number of cells in the monolayer that have the insulin promoter turned on. Thus, nicotinamide can be used to induce the cells to produce insulin. Finally, FIG. 10 confirms by RT-PCR that the HPDE6c7 cells are producing insulin mRNA at least when the cells are incubated in keratinocyte serum-free medium (lanes 9 through 14). Thus, the above results indicate that the HPDE6c7 can be induced to differentiate into insulin-producing cells.

Thus, as shown herein, the HPDE6c7 cells of the present invention include a population of cells that retain pluripotent stem cell characteristics and thus have the potential to be induced to become insulin-producing cells. As shown herein, under specific culture conditions, the HPDE6c7 cells produced insulin (as measured in the medium) and showed insulin gene expression. Thus, the HPDE6c7 cells can be induced to differentiate into endocrine cells with the ability to produce insulin and secrete insulin in a regulated manner under the appropriate conditions for enrichment or proliferation.

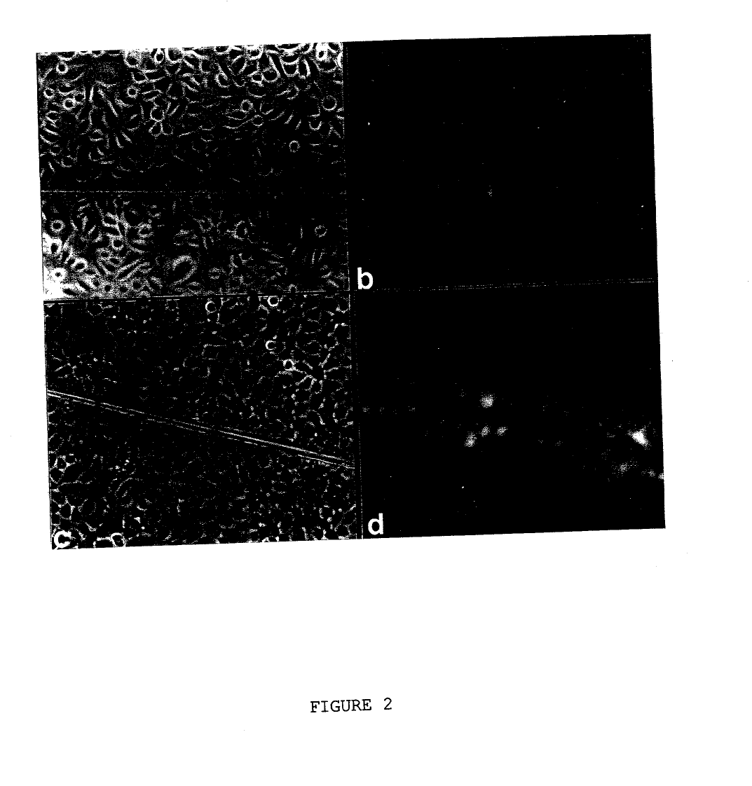

As shown in FIG. 2A, the HPDE6c7 cells form ductal-like structures with budding structures in MATRIGEL which are similar in appearance to the cultivated human islet buds (CHIBs) disclosed in Bonner-Weir et al., Proc. Natl. Acad. Sci. USA 97: 7999-8004 (2000). CHIBs were observed to bud off of duct-like structures derived from human pancreatic duct tissue that had been propagated in cell culture as monolayers with an epithelial morphology and then grown in MATRIGEL wherein the cells differentiated into ductal-like structures. The CHIBs were shown to accumulate zinc and to express insulin in response to glucose. Therefore, growing the HPDE6c7 cells in MATRIGEL can induce the HPDE6c7 cells to differentiate into ductal-like structures wherein the budding structures have the capacity to differentiate into endocrine cells such as insulin-producing cells and other hormone-producing cells. These budding structures are derivatives of the HPDE6c7 which can be used as a source of cells for producing insulin in vitro or as a source of cells for use in artificial pancreata to be transplanted into diabetic mammals. Alternatively, the HPDE6c7 may be induced to differentiate into insulin-producing cells by incubating the cells on plastic cell culture dishes or MATRIGEL in media such as complete or basal keratinocyte media containing about 10 mM nicotinamide or media with or without nicotinamide containing about 5% human serum, preferably supplemented with 10 to 25 mM glucose, and optionally containing one or more of the biological factors including, but not limited to, hepatocyte growth/scatter factor, insulin-like-growth factor, epidermal growth factor, keratinocyte growth factor, fibroblast growth factor, and other factors which modulate cellular growth. The HPDE6c7 cells can then be used as a source for insulin in vitro or as a source of cells for artificial pancreata. Using HPDE6c7 cells to produce differentiated insulin-producing cells is an improvement over the cells disclosed in Bonner-Weir et al. because the HPDE6c7 cells are immortal which enables them to be expanded indefinitely to produce large quantities of cells using commercial cell production technologies. Therefore, the HPDE6c7 cells can provide sufficient quantities of differentiated insulin-producing cells to enable artificial pancreas transplantation therapies to become a viable alternative to current treatments for insulin-dependent diabetes. In contrast to the HPDE6c7 cells, the Bonner-Weir et al. cells are mortal and produce only a limited amount of CHIBs; therefore, a continuous source of fresh pancreatic duct tissue is required to produce sufficient quantities of CHIBs.

Thus, the HPDE6c7 cells or derivative thereof can be used to obtain a molecular understanding of the genes that are needed for the development and differentiation of pancreatic cells into insulin-producing cells, to determine how these pancreatic cells can form three-dimensional “organoids,” to use as a means to screen agents that induce or inhibit differentiation of insulin-producing cells and tissues, to study pancreatic cell carcinogenesis, and to use in therapies that replace insulin in diabetic patients.

While isolating primary islet or beta cells from animal or human pancreata can be used for these purposes, the cost, inconvenience of isolating the islet cells each time for every use, the inability to obtain adequate supplies of human pancreata, and in the case of animals, cross-species problems of extrapolating results to human pancreatic development, diabetes, or pancreatic diseases, have made it desirable to find an alternative to primary islet or beta cells. The HPDE6c7 cells provide an alternative to primary islet or beta cells. A useful property of the HPDE6c7 cells or derivative thereof is that they can be maintained or propagated as immature stem cells. When insulin-producing cells are needed, the cells are induced to differentiate into insulin-producing cells. Therefore, because the HPDE6c7 cells can be induced to differentiate into insulin-producing cells and form organoids, the HPDE6c7 cells are a valuable resource for the pharmaceutical, bioengineering, and tissue engineering companies.

The HPDE6c7 cells or derivative thereof are particularly useful for preparing artificial pancreata, which can then be used in transplants for treating patients with IDDM Type 1 diabetes, for methods for identifying chemicals or conditions that can induce or inhibit differentiation of pancreatic stem cells into particular differentiated pancreatic cells, methods for identifying chemicals or conditions that can induce or inhibit malignancy in pancreatic cells, and methods for developing treatments for pancreatic cancers.

### (a) Artificial Pancreata for Treating IDDM Type 1 Diabetes

Means for encapsulating cells that can be used as an artificial pancreas are known in the art. These means can be used to encapsulate the HPDE6c7 cells or derivative thereof under conditions that induce the cells to produce insulin in response to external stimuli or to produce insulin constitutively. Preferably, the HPDE6c7 cells are induced to differentiate into insulin-producing beta cells in vitro which are then are encapsulated in a device to produce an artificial pancreas. The following U.S. patents disclose devices which can provide a means for producing an artificial pancreas comprising the HPDE6c7 cells or derivative thereof.

U.S. Pat. No. 6,023,009 to Stegemann et al. discloses an artificial pancreas that comprises one or more pancreatic islet cells capable of producing insulin, encapsulated within an agar gel bead, wherein each bead can be installed within a diffusion chamber or perfusion chamber that enables insulin, glucose, and nutrient transport by diffusion or perfusion. A particularly desirable device for holding the above beads is the perfusion device disclosed therein that comprises a hollow fiber that has one end connected to a blood vessel for receiving blood and another blood vessel for returning the blood. The beads containing the pancreatic islet cells are seeded around the hollow fiber and the entire device encapsulated in an acrylic housing having a pore size to protect the cells from immune reactive materials. Instead of the islet cells, the HPDE6c7 cells or derivative thereof can be incorporated into the above agar gel beads which can then be used in the above device. Other encapsulation methods such as those disclosed in U.S. Pat. No. 5,980,889 to Butler et al. or U.S. Pat. No. 5,997,900 to Wang et al. are also suitable for encapsulating the HPDE6c7 cells or derivative thereof.

U.S. Pat. No. 5,702,444 to Struthers et al. discloses an artificial pancreas comprising a reactive body of soft, plastic, biocompatible, porous hydratable material containing therein a multiplicity of islet cells. Each of the islet cells are jacketed in a hydrogel gum preferably selected from the group consisting alginates, guar gums, agars, agaroses, and carrageens. The jackets about the islet cells are in bridging contact with each other and support the islet cells in a predetermined spaced relationship from each other in a matrix comprising a suitable water-soluble polymer such as hydroxy celluloses, polyvinyl alcohols, polyvinyl pyrrolidones, etc. to make the body. The above body is then enveloped and supported by a microporous barrier membrane preferably comprising a cellulosic derivative such as regenerated cellulose and cellulose acetates, cellulose esters or ethers or acrylates, etc., in spaced relationship to the islet cells therein and through which molecules greater than 60,000 Daltons cannot move. Instead of the islet cells, the HPDE6c7 cells or derivative thereof can be used to make the above artificial pancreas. Preferably, the HPDE6c7 cells are induced to differentiate into beta cells in vitro which are then encapsulated in the artificial pancreas.

U.S. Pat. Nos. 5,425,764 and 5,855,616, both to Fournier et al. disclose an implantable artificial pancreas having a chamber containing insulin-secreting islet cells, one or more vascularizing chambers open to surrounding tissue, a semi-permeable membrane between the islet chamber and the vascularizing chamber that allows passage of small molecules such as insulin, glucose, and oxygen and does not allow immunogenic agents to pass. The vascularizing chamber contains growth factor soaked fibrous or foam matrix having a porosity of about 40 to 95%, which allows small capillary growth and prevents blood clotting. Instead of the islet cells, the HPDE6c7 cells or derivative thereof can be used to make the above artificial pancreas. Preferably, the HPDE6c7 cells are induced to differentiate into beta cells in vitro which are then encapsulated in the artificial pancreas.

U.S. Pat. No. 5,741,334 to Mullon et al. discloses an artificial pancreatic perfusion device comprising a hollow fiber that has one end connected to a blood vessel for receiving blood and a second end connected to a blood vessel for returning the blood. The islet cells surround the hollow fiber and the hollow fiber and islet cells are surrounded by a housing comprising a semipermeable membrane having a pore size small enough to offer protection to the islets and host from immune reactive substances. The HPDE6c7 cells or derivative thereof can be used in place of the islet cells to provide the artificial pancreas. Preferably, the HPDE6c7 cells are induced to differentiate into beta cells in vitro which are then encapsulated in the artificial pancreas.

U.S. Pat. Nos. 5,855,613 and 6,165,225, both to Antanavitch et al., disclose an artificial pancreas comprising islet cells in high-density-cell in thin sheets comprising a purified biocompatible gelled alginate which does not produce any significant foreign body reaction or fibrosis. The HPDE6c7 cells or derivative thereof can be used in place of the islet cells to provide the artificial pancreas. Preferably, the HPDE6c7 cells are induced to differentiate into beta cells in vitro which are then encapsulated in the artificial pancreas.

The methods disclosed in U.S. Pat. Nos. 5,795,790, 5,840,576, 5,843,431, 5,853,717, 5,858,747, and 5,935,849, all to Schinstine et al., disclose methods for controlling cell distribution, proliferation, differentiation, and gene expression in artificial organs. The methods disclosed therein can be used to control the distribution, proliferation, differentiation, and gene expression of HPDE6c7 cells or derivative thereof in an artificial pancreas.

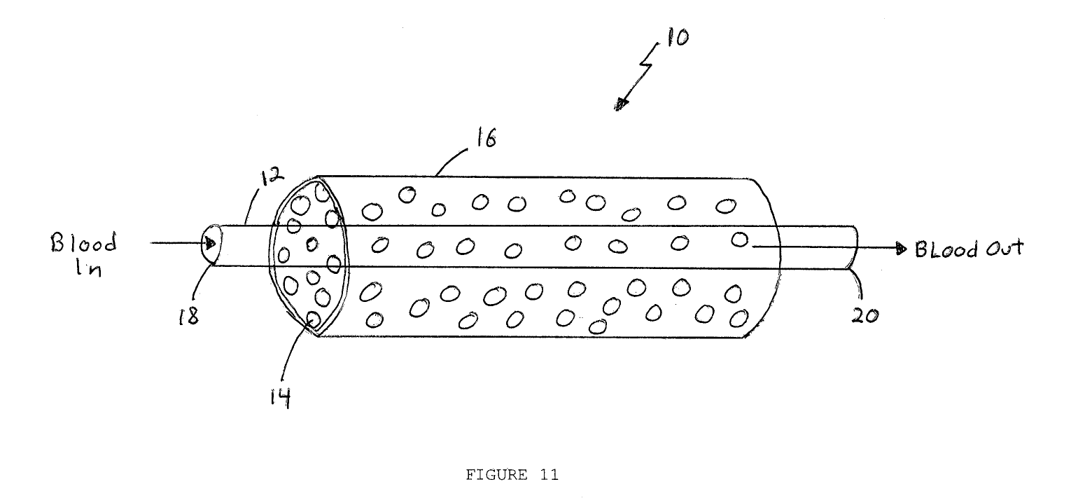

FIG. 11, by way of illustration only, shows a schematic representation of one embodiment of an artificial pancreas that can be used with the HPDE6c7 cells or derivative thereof for transplantation into a mammal for the therapy of IDDM. Artificial pancreas 10 comprises hollow fiber 12, HPDE6c7 cells or derivative thereof 14, and housing 16. Blood enters inlet end 18 and exists outlet end 20. Hollow fiber 12 comprises a porous polymer that restricts the entry into the artificial pancreas 10 of immune reactive molecules and cells while allowing entry of nutrients and glucose and exit of insulin and waste products produced by the cells. Housing 16 comprises any biocompatible material and is preferably a semi-permeable membrane that protects HPDE6c7 cells or derivative thereof 14 from the host's immune reactive molecules and cells. Preferably, the HPDE6c7 cells are induced to differentiate into beta cells in vitro which are then encapsulated in the artificial pancreas. The amount of insulin artificial pancreas 10 can produce is dependent on the number of HPDE6c7 cells or derivative thereof 14 which is in turn dependent on the surface area of hollow fiber 12. The greater the surface area of hollow fiber 14, the greater the number of HPDE6c7 cells or derivative thereof 14 that can be contained in artificial pancreas 10. Artificial pancreas 10 is connected at inlet end 18 and outlet end 20 to a blood vessel to allow continuous blood flow through hollow fiber 12. The blood vessel can be an artery or a vein; however, an artery to vein connection is preferred.

### (b) Method for Identifying Chemicals or Conditions that Induce or Inhibit Differentiation of Pancreatic Cells

The HPDE6c7 cells or derivative thereof are useful in methods for determining whether a chemical, drug, or particular culture conditions can induce or inhibit differentiation of pancreatic stem cells. For example, nicotinamide and cholera toxin can induce the HPDE6c7 cells to differentiate into cells that produce insulin and sonic hedgehog can turn on the promoter for the insulin gene in HPDE6c7 cells. Other chemicals such as those in tobacco smoke or environmental chemicals can inhibit differentiation. Thus, the HPDE6c7 cells are useful for screening chemical agents to identify chemicals which may induce or inhibit pancreatic carcinomas, diabetes, or other pancreatic diseases in vitro.

To determine whether a chemical can induce or inhibit differentiation, HPDE6c7 cells or derivative thereof are seeded to a series of wells in a tissue culture plate at about 30 to 50% confluence or at about 5 to 10×10⁵ cells per well in a medium such as KBM at 37° C. for about 30 minutes. Preferably, at least one well is not incubated with the chemical. The cells are continued to be incubated at 37° C. In particular embodiments, the chemical is added to the cells with each medium change. At particular time points, the ability of the chemical to induce differentiation of the cells is determined by measuring GJIC using the Lucifer yellow dye transfer method as taught by El-Fouly et al. (Exp. Cell Res. 168: 422-430 (1987)) or by visual observation. In particular embodiments, the HPDE6c7 cells are cultured in a medium comprising a three-dimensional matrix such as a collagen-based medium. An example of a collagen-based medium is MATRIGEL, a commercial preparation of murine basement membrane (Collaborative Research, Inc., Waltham, Mass.).

### (c) Method for Identifying Chemicals that can Induce or Inhibit Malignant Proliferation of Pancreatic Cells

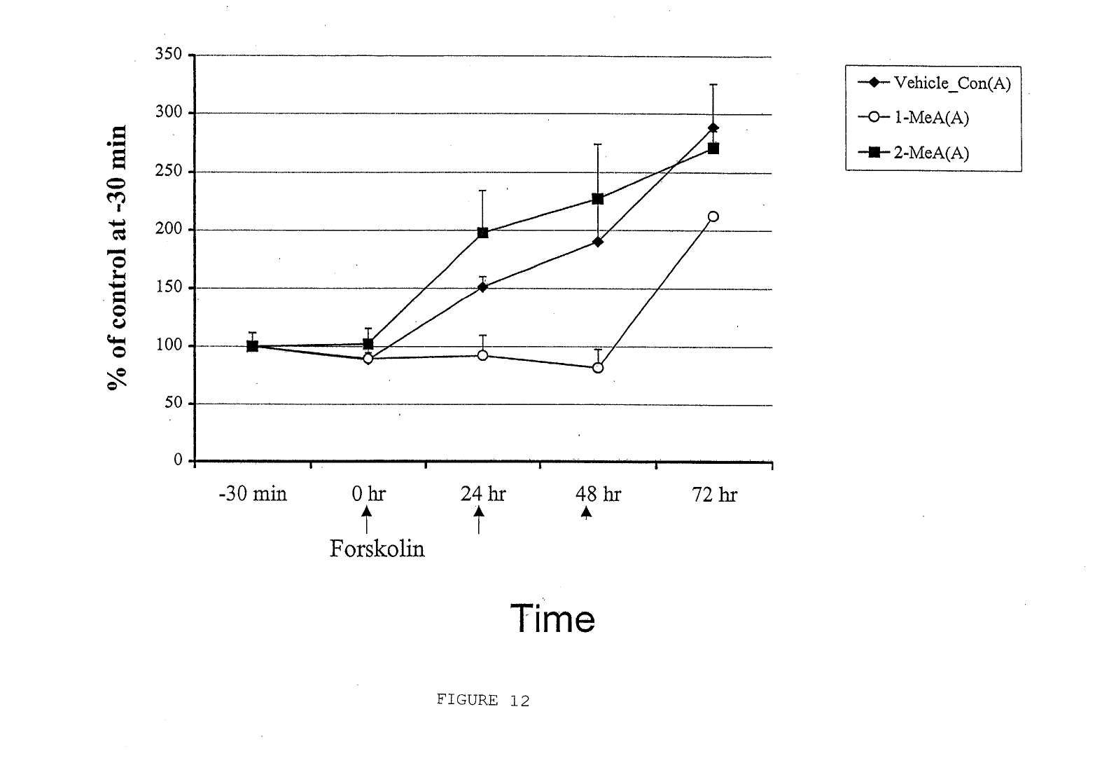

The HPDE6c7 cells or derivative thereof are useful in methods for determining whether a chemical, drug, or particular culture conditions can induce malignant proliferation or inhibit malignant proliferation of pancreatic cells, in particular, pancreatic stem cells. Particular chemicals can induce the HPDE6c7 cells to proliferate. For example, culturing the HPDE6c7 cells in the presence of hepatic growth factor or beta-cellulin causes an increase in growth of the cells. Cigarette smoke has been implicated as a cause for some forms of pancreatic cancer. Incubation of the HPDE6c7 cells in the presence of 1-methylanthracine, which is a carcinogenic constituent of cigarette smoke, inhibited GJIC that had been induced by forskolin. In contrast, 2-methylanthracine, which is an analog of 1-methylanthracine that is not carcinogenic, did not. These results are shown in FIGS. 12. FIG. 8 further shows that the inhibitory effect of 1-methylanthracine is reversible and not cytotoxic. These results demonstrate that the HPDE6c7 cells or derivative thereof are useful for methods that determine whether a particular chemical can cause pancreatic cells or stem cells to become malignant. The assay measures the ability or inability of a chemical to inhibit the GJIC in the cells after GJIC has been induced by forskolin or other c-AMP elevating chemical.

To determine the ability of a chemical to induce malignant proliferation in pancreatic cells, in particular, pancreatic stem cells, HPDE6c7 cells or derivative thereof are seeded to a series of wells in a tissue culture plate at about 30 to 50% confluence or at about 5 to 10×10⁵ cells per well in a medium such as KBM containing a particular concentration of the chemical at 37° C. for about 30 minutes. Afterwards, forskolin is added to a final concentration of about 5 μM to induce GJIC in the cells. Preferably, at least one well is not incubated with the chemical. The cells are continued to be incubated at 37° C. In particular embodiments, forskolin is also added to the cells 24 hours and 48 hours after the above chemicals were initially added to the cells. The chemical is added to the cells with each medium change. At particular time points, the effect of the chemical on the cells is determined by measuring GJIC using the Lucifer yellow dye transfer method as developed by El-Fouly et al. (Exp. Cell Res. 168: 422-430 (1987)) or by visual observation. An inhibition of GJIC indicates that the chemical is capable of inducing proliferation of pancreatic cells. In particular embodiments, the HPDE6c7 cells are cultured in a medium comprising a three-dimensional matrix such as the collagen-based medium MATRIGEL. In particular embodiments, a control is provided wherein the chemical is added only at the time the cells are seeded to the plates. By adding the chemical only at time the cells are seeded, it can be determined whether the chemical is inhibiting GJIC induced by forskolin or inhibiting the effect of forskolin, or whether the chemical is cytotoxic to the cells.

To determine the ability of a chemical to inhibit the effect of a GJIC-inhibiting chemical that can induce proliferation of pancreatic cells or stem cells, HPDE6c7 cells or derivative thereof are seeded to a series of wells in a tissue culture plate at about 30 to 50% confluence or at about 5 to 10×10⁵ cells per well in a medium such as KBM containing a particular concentration of the GJIC-inhibiting chemical at 37° C. for about 30 minutes. Afterwards, forskolin is added to a final concentration of about 5 μM to induce GJIC. Preferably, at least one well is not incubated with the chemical. Next, a particular concentration of the chemical to be tested is added to the wells. Preferably, at least one well is not incubated with the chemical. The cells are continued to be incubated at 37° C. At each medium change, the GJIC-inhibiting chemical and the chemical being tested are added to the cells. In particular embodiments, forskolin can also be added to the cells 24 hours and 48 hours after the above chemicals were initially added to the cells. At particular time points, the effect of the chemical being tested on abrogating the effect of the GJIC-inhibiting chemical on the cells is determined by measuring GJIC using the Lucifer yellow dye transfer method as developed by El-Fouly et al. (Exp. Cell Res. 168: 422-430 (1987)) or by visual observation. Establishment of GJIC in the cells in the wells containing both the GJIC-inhibiting chemical and the chemical being tested in the presence of forskolin indicates that the chemical has an inhibitory effect on the GJIC-inhibiting chemical's ability to induce proliferation of pancreatic cells or stem cells. The lack of GJIC in the cells in those wells containing both the GJIC-inhibiting chemical and the chemical being tested in the presence of forskolin indicates that the chemical has no inhibitory effect on the GJIC-inhibiting chemical's ability to induce proliferation of pancreatic cells or stem cells. In particular embodiments, the HPDE6c7 cells are cultured in a medium comprising a three-dimensional matrix such as a collagen-based medium such as MATRIGEL.

The following examples are intended to promote a further understanding of the present invention.

### EXAMPLE 1

The human pancreatic ductal epithelial clone 7 (HPDE6c7) cell line is a clonal population of cells derived from the pancreas of a 77-year-old male, which were immortalized by Furukawa et al. (Amer. J. Path. 148: 1763-1770 (1996)) by infecting the cells with an amphotrophic retrovirus containing human papilloma virus (HPV) 16 genes E6 and E7. The HPDE6c7 cells were routinely cultured in complete keratinocyte serum-free medium (KSFM) containing insulin (less than mg/L), hydrocortisone (less than 0.1 mg/L), epidermal growth factor (EDF) (5 ng/ml), and bovine pituitary extract (BPE) (50 mg/ml) at 37° C. in a 5% CO₂ atmosphere. Complete KSFM containing growth factors, hormones, and bovine pituitary extract was purchased from Life Technologies, Grand Island, N.Y. The HPDE6c7 cells were at times also incubated in keratinocyte basal medium (KBM) without growth factors or hormones, which was also purchased from Life technologies. In general, the cells were routinely passed in culture by dissociating the cells from cell culture dishes or cell culture wells when the cells had grown to about 80 to 90% confluence with trypsin-EDTA and replating to new cell culture dishes or wells at a density of about 30 to 50% confluence or at about 5 to 10×10⁵ cells per well.

### EXAMPLE 2

Growth of the HPDE6c7 cell line in tissue culture as a monolayer was compared to its growth in MATRIGEL, a collagen gel-based medium.

To study the growth response of the HPDE6c7 cells, the cells were first cultured on plastic tissue culture dishes in KSFM. Afterwards, the cells were removed by trypsinization using trypsin-EDTA and seeded at high density (30 to 40% confluence) on growth factor-free MATRIGEL (about 0.2 to 0.4 ml of trypsinized cells per well) in 24-well tissue culture dishes. MATRIGEL was purchased from Collaborative Research, Inc., Waltham, Mass. At different times after plating, photographs were taken of the growth and three-dimensional organization of the cells in the cultures.

When cultured as a monolayer on plastic cell culture dishes, the cells showed a morphology characteristic of epithelial cells: a cobblestone appearance (FIG. 1A). The cells were also contact-inhibited at confluence and did not show multilayered growth indicating that immortalizing the cells as in Example 1 did not neoplastically transform the cells. When the cells were plated on MATRIGEL, the cells showed marked changes in their pattern of growth. Within 24 hours after plating on MATRIGEL, the cells organized into tubular/ductal structures that showed a networking pattern with extensive branching and budding patterns (FIG. 1B). The growth of this branching network was three-dimensional and extended into the MATRIGEL. The ductal and budding structure growth of the cells was maintained for several days after plating on MATRIGEL.

### EXAMPLE 3

The gap junctional intercellular communication (GJIC) competence of the HPDE6c7 cells was determined and the effect of increasing the level of c-AMP in the cells was determined.

The ability of the HPDE6c7 cells to communicate via gap junctions under various growth conditions was measured using the lucifer yellow dye transfer method as developed by El-Fouly et al. (Exp. Cell Res. 168: 422-430 (1987)). Briefly, near confluent cultures of the cells grown as in Example 1 were subjected to the desired treatment, either growth in KBM or KBM containing c-AMP elevating agents such as forskolin or forskolin and 3-isobutyl-1-methylxanthine (IBMX), both from Sigma Chemical Co., St. Louis, Mo. Then, lucifer yellow was loaded into the cells by making two or three scrape lines in the monolayer with a sharp scalpel. In GJIC competent cells, lucifer yellow moves through gap junctions from the primary dye loaded cells to contacting neighbors whereas in GJIC incompetent cells, the dye does not transfer from the primary dye loaded cells to the neighboring cells. Quantifying the extent of communication was done using an image analysis program.

When the HPDE6c7 cells were cultured in complete growth medium (KSFM, which contains growth factors, hormones, and bovine pituitary extract as in Example 1), the cells were GJIC incompetent (FIGS. 2A and 2B). When the cells were cultured in KBM, which lacks growth factors, hormones, and pituitary extract, there was a significant increase in the level of their GJIC at 48 hours. Treating the cells with agents that elevated the level of c-AMP in the cells (forskolin and IBMX) for 48 hours also significantly enhanced the extent of GJIC of these cells compared to cells grown in complete growth medium (FIGS. 2C, 2D, and 3). GJIC of the cells increased when the cells were cultured in KBM for 48 hours. The increase in GJIC of the cells in KBM or in KBM containing forskolin or forskolin and IBMX was very similar relative to those in complete keratinocyte serum-free medium (KSFM), 2.1- and 2.2-fold increases, respectively (FIG. 3). The c-AMP elevating agents also increased GJIC when the cells were maintained in complete KSEM by 50%. The results confirm that the HPDE6c7 cells are stem cell-like and further shows that the HPDE6c7 cells are capable of being induced to differentiate into particular pancreatic cells such as insulin-producing beta cells.

### EXAMPLE 4

In this example, the gap junction proteins expressed by the HPDE6c7 cells and the effect of elevating the level of c-AMP in the cells on the expression of the genes encoding the gap junction proteins was determined by Western blotting.

HPDE6c7 cells that had been grown in tissue culture dishes under particular growth conditions were lysed in a buffer containing 62.5 mM Tris-HCl, pH 7.4, 20% SDS, 5 mM EDTA, 2 mM PMSF, and 10 μg/ml leupeptin. Cells that had been grown on MATRIGEL were lysed as follows. The cultures were incubated with about 2 ml MATRISPERSE (Collaborative Research, Inc.) at 4° C. for a few hours to dissolve the MATRIGEL and release the cells. The cells were collected by low-speed centrifugation, washed in PBS, and re-centrifuged. The protein concentrations of the samples were determined using a Bio-Rad DC protein assay kit (Bio-Rad Laboratories, Hercules, Calif.). Fifty μg of total cellular proteins of each sample was loaded onto 10 or 12.5% SDS-polyacrylamide gels and the proteins in the samples resolved by electrophoresis. The resolved proteins were transferred to nitrocellulose membranes as taught by Laemmli (Nature (Lond.) 227: 680-685 (1970) and Tobin et al. (Proc. Natl. Acad. Sci. USA 76: 4350-4354 (1978)). The immunological detection of the gap junction proteins was performed using antibodies against connexin (Cx) 43, 45, 32, and 26 and an enhanced chemiluminescence (ECL) detection method (Fischer et al., Toxicol. Appl. Pharmacol. 159: 194-203 (1999)). The antibodies against Cx43, 32, and 26 were purchased from Zymed Laboratories, Inc., South San Francisco, Calif., and antibodies against Cx45 was purchased from Alpha Diagnostic International, Inc., San Antonio, Tex. The ECL reagents and HYBOND film was purchased from Amersham Pharmacia Biotech, Piscataway, N.J.

Cx43 was the only gap junction protein expressed in the HPDE6c7 cells with or without elevating the level of c-AMP in the cells (FIG. 4). Elevating intracellular c-AMP levels by treating the cells with forskolin and IBMX significantly increased the levels of Cx43 protein in a time dependent manner (FIG. 4). Expression of Cx26, Cx32, or Cx45 under similar growth conditions was not seen. Cx43 gap junction protein was also increased when the cells were grown on MATRIGEL in the presence of c-AMP elevating agents (data not shown)

### EXAMPLE 5

In this example, RT-PCR was used to characterize the types of gap junction genes that are expressed in the HPDE6c7 cells.

HPDE6c7 cells were incubated in KBM for 4 days, KBM containing c-AMP elevating agents (forskolin and IBMX) for 48 hours, KGM (keratinocyte growth medium which is the equivalent of KSFM) containing 10 mM nicotinamide for 4 days, KGM containing 100 μM dibutyryl c-AMP (c-AMP) for 4 days, or KGM containing 10 mM nicotinamide and 100 μM c-AMP for 4 days. Afterwards, total RNA was isolated from the cells using TRIZOL reagent (GIBCO BRL, Gaithersburg, Md.). The total RNA (about 0.5 μg) was then incubated at 37° C. for 10 minutes with 2 units of DNASE 1 (Roche Molecular Biochemical, GmBH, Germany) and 2 units of RNase inhibitor (Roche Molecular Biochemical, GmBH). Afterwards, the total RNA was heated to 75° C. for 10 minutes to heat inactivate the DNase 1. RT-PCR was then performed using the Titan TM One Tube RT-PCR System (Roche Molecular Biochemicals, GmBH) according to the manufacturer's instructions using 10 pmoles of the following oligodeoxynucleotide PCR primer pairs. To amplify Cx45, sense primer 5′-GGAGCACGCTGAAGCAGAC-3′ (SEQ ID NO:1) and antisense primer 5′-CGGGTGGACTTGGAAGCCA-3′ (SEQ ID NO:2) (Chanson et al., J. Clin. Invest. 130: 1677-1684 (1999). As a control, β-actin was amplified using sense primer 5′-CGGCATCGTCACCAACTGGGA-3′ (SEQ ID NO:3) and antisense primer 5′-CGTAGATGGGCAGTGTGGG-3′ (SEQ ID NO:4). The Cx45 primers allow a 309 bp PCR product to be amplified and the β-actin primers allow a 280 bp PCR product to be amplified. The total RNA was made into cDNA by according to the manufacturer's instruction accompanying the Titan One Tube RT-PCR System. Next, the amplification was performed for 35 cycles, each comprising 30 seconds at 94° C., 30 seconds at 60° C., and 30 seconds at 68° C. using a GENEAMP PCR System 9700 (Perkin Elmer, Norwalk, Conn.). After the last cycle, an elongation step for 5 minutes at 68° C. was performed. The PCR products were resolved on 1.5% agarose gels and visualized using ethidium bromide staining. RT-PCR amplification of Cx26, 32, and 43 is described in Trosko et al., Methods 20: 245-264 (2000).

As shown in FIG. 5, there was an increase in the steady-state level of Cx43 gene expression after growing the HPDE6c7 cells in KBM or KGM containing c-AMP elevating agents compared to the cells grown in complete growth medium. The analysis, which was not by quantitative RT-PCR, was replicated (data not shown). RT-PCR also showed that growing the HPDE6c7 cells with c-AMP elevating agents caused expression of the Cx32 gene but not the Cx26 gene. However, the cells did not produce detectable levels of the Cx32 protein.

As shown in FIG. 6, the Cx45 gene was expressed under all of the above growing conditions. Lanes 2 and 7 show the RT-PCR product for HPDE6c7 cells incubated in KBM, lanes 3 and 8 show the RT-PCR product for HPDE6c7 cells incubated in KGM containing 10 mM nicotinamide, lanes 4 and 9 show the RT-PCR product for HPDE6c7 cells incubated in KGM containing 100 μM c-AMP, and lanes 5 and 10 show the RT-PCR product for HPDE6c7 cells incubated in KGM containing 10 mM nicotinamide and 100 μM c-AMP.

RT-PCR detection Cx36 gene expression in HPDE6c7 cells treated with c-AMP elevating agent in KBM from 24 to 72 hours or with forskolin for 72 hours was as follows. The two primer sequences of human Cx36 were the sense primer 5′ACGCCGCTACTCTACAGTCTTCC-3′ (SEQ ID NO:7) and the antisense primer 5′-GATGCCTTCCTGCCTTCTGAGCTT-3′ (SEQ ID NO:8). Glyceraldehyde 3-phosphate dehydrogenase (GAPDH) expression was measured as an internal standard. The primers were the sense primer 5′GTTCGACAGTCAGCCGCATC-3′ (SEQ ID NO:9) and the antisense primer 5′-GTGGGTGTCGCTGTTGAAGTC-3′ (SEQ ID NO:10). C-DNA was made as above.

For the PCR reaction, MgCl₂ (50 mM) was added to 5 μl Cx36 cDNA (1:5 dilution with DEPC-treated water from earlier preparation) for a final concentration of 1.5 mM along with 5 μl 10× PCR buffer (200 mM Tris-HCL, pH 8.4, 500 mM KCl), 1 μl of each 10 mM dNTP, AMPLITAQ GOLD polymerase (2 units, Perkin Elmer), and 5 pmol of sense and antisense primer in 50 μl. Next, the mixture was first heated at 94° C. for five minutes in a PTC-200 Engine Thermal-Cycler (MJ Research, Waltham, Mass.).

Amplification of Cx36 and GAPDH were performed in 35 cycles at 94° C. for one minute, 63° C. for one minute, and 72° C. for two minutes, and 94° C. for 45 seconds, 60° C. for 45 seconds, and 72° C. for two minutes. The predicted amplified sizes of the Cx36 and GAPDH cDNA amplified products were 269 and 933 bp, respectively. The PCR products were analyzed on 1.5% agarose gels in 0.5× Tris borate/EDTA buffer and stained with cyber green.

The results (not shown) showed that Cx36 expression was detected after 72 hours incubation with forskolin but not when treated with c-AMP elevating agent in KBM from 24 to 72 hours.

### EXAMPLE 6

This example demonstrates that the HPDE6c7 cells can be used in a method for determining whether a chemical is capable of inducing malignant proliferation of pancreatic cells or stem cells. The method measures the ability or inability of a chemical to inhibit the GJIC in the cells after it had been induced by forskolin.

An assay comprising the HPDE6c7 cells was tested with the polycyclic aromatic hydrocarbons (PAH) 1-methylanthracine, a known carcinogen present in tobacco smoke, and 2-methylanthracine, an analog of 1-methylanthracine that is not a carcinogen, to determine whether the chemicals would inhibit GJIC. The method was able to identify that 1-methylanthracine inhibited GJIC whereas 2-methylanthracine and the carrier for the chemicals did not.

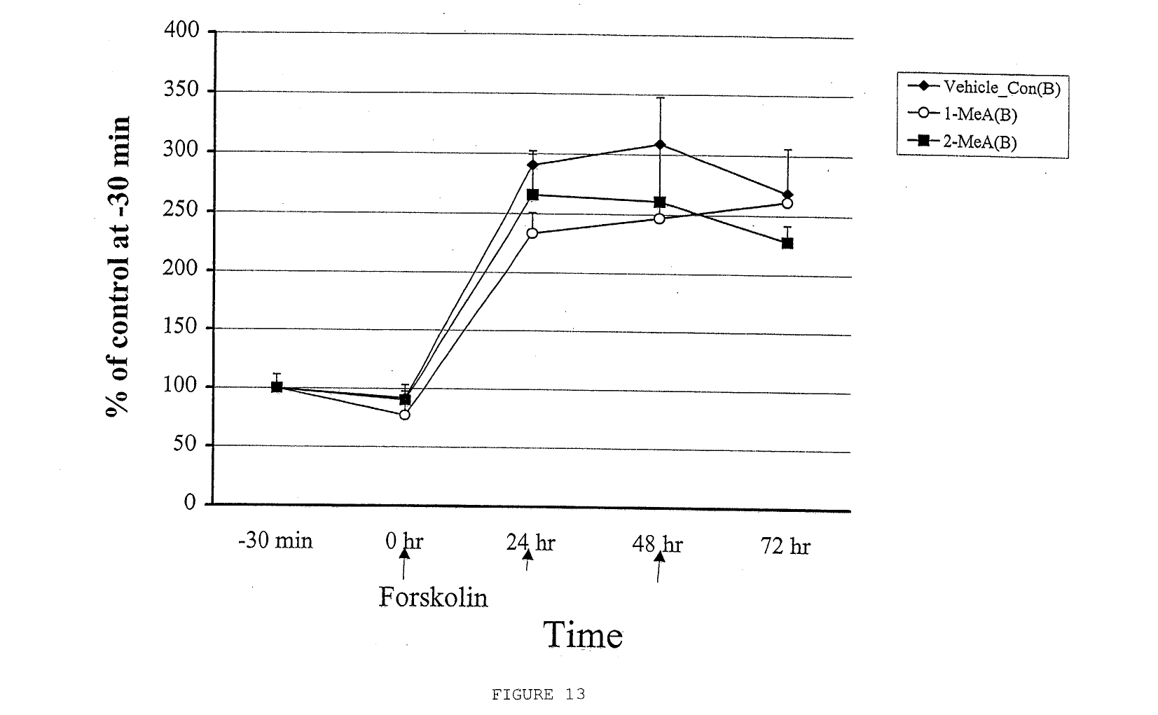

Two separate experiments were performed, each as follows. For each experiment, the HPDE6c7 cells were seeded to a series of wells in a tissue culture plate at a density of about 30 to 50% confluence or about 5 to 10×10⁵ cells per ml and grown in KBM containing either 1-methylanthracine (1-MeA) in the PAH carrier acetonitrile (1-MeA final concentration in KBM was 70 μM), 2-methylanthracine (2-MeA) in the PAH carrier acetonitrile (2-MeA final concentration in KBM was 70 μM), or the PAH carrier (Vehicle_Con) (final concentration in KBM was 0.7%) at 37° C. for 30 minutes. After 30 minutes, forskolin was added to a final concentration of 5 μM. The incubation of the cells was continued at 37° C. Additional forskolin was added to the cells 24 hours and 48 hours after the above chemicals had been added. GJIC was measured as described in Example 3 at time of addition of the above chemicals, and again at 24, 48, and 72 hours post-addition. The medium was changed every 24 hours. In assay (A) 1-methylanthracine, 2-methylanthracine, or the PAH carrier was given at every medium change whereas in assay (B), 1-methylanthracine, 2-methylanthracine, or the PAH carrier was given only at the time of the cells were seeded. The results for assay (A) are shown in Table 1 and FIG. 12 and the results for assay (B) are shown in Table 2 and FIG. 13

TABLE 1
Average (Percent standard deviation)

| | −30 min | 0 hr | 24 hr | 48 hr | 72 hr |
|---|---|---|---|---|---|
| Vehicle_Con | 100.00 | 88.57 | 151.12 | 190.18 | 288.56 |
| | (11.58) | (6.13) | (8.94) | (38.87) | (37.18) |
| 1-MeA | 100.00 | 89.37 | 92.24 | 81.77 | 212.39 |
| | (11.58) | (8.87) | (17.32) | (16.08) | (0.10) |
| 2-MeA | 100.00 | 102.06 | 197.60 | 227.15 | 271.19 |
| | (11.58) | (13.20) | (36.49) | (47.06) | (13.88) |

Vehicle_Con is the PAH carrier. 1-MeA is 1-methylanthracine. 2-MeA is 2-methylanthracine. PAH was given at every medium change.

TABLE 2
Average (Percent standard deviation)

| | −30 min | 0 hr | 24 hr | 48 hr | 72 hr |
|---|---|---|---|---|---|
| Vehicle_Con | 100.00 | 91.61 | 290.41 | 308.65 | 268.46 |
| | (11.58) | (6.09) | (34.44) | (39.60) | (21.51) |
| 1-MeA | 100.00 | 77.04 | 233.32 | 246.93 | 261.32 |
| | (11.58) | (8.87) | (23.42) | (40.22) | (18.76) |
| 2-MeA | 100.00 | 89.95 | 265.70 | 260.89 | 260.89 |
| | (11.58) | (17.05) | (11.26) | (30.16) | (2.09) |

Vehicle_Con is the PAH carrier. 1-MeA is 1-methylanthracine. 2-MeA is 2-methylanthracine. PAH was given at time −30 min only.

Table 1 and FIG. 12 show that 1-methylanthracine inhibited GJIC induced by forskolin whereas 2-methylanthracine and the PAH carrier did not. Since chemicals that inhibit GJIC in other cell types have been shown to be tumor promoters, the results imply that the 1-methylanthracine might contribute to the promotion of pancreatic cancer.

Table 2 and FIG. 13 show that 1-methylanthracine had no lethal effects on the cells and that the inhibition of GJIC caused by 1-methylanthracine was reversible. It also showed that its inhibitory effect was only after GJIC had been induced by the forskolin and not on the ability of forskolin to induce GJIC.

These results demonstrate the utility of the HPDE6c7 cells or derivatives thereof in a method for screening chemicals or drugs that might contribute to pancreatic cancer. Alternatively, the cells can be used to screen for chemicals that inhibit the effect of chemicals that can induce of pancreatic cancer.

### EXAMPLE 7

This example demonstrates that the HPDE6c7 cells are immortalized pancreatic stem cells with the potential to differentiate into insulin-producing cells.

Insulin complexes with zinc, therefore, the HPDE6c7 were assayed for the ability to accumulate zinc. The HPDE6c7 cells were grown on plastic cell culture dishes in KBM to produce a cell monolayer. Then the cells were stained with dithizone as developed by Latif et al. (Transplantation 45: 827-830 (1988)). FIG. 7 shows that when the cell monolayer was stained with dithizone, a reagent that stains for zinc, particular cells in the monolayer were stained indicating that the cells accumulated zinc. This result indicates that cells accumulate zinc and, therefore, have the potential to produce insulin.

To determine whether the insulin promoter in the HPDE6c7 cells is active, the cells were infected with adenovirus AdINSGFP, a virus vector that expresses the Green Fluorescence Protein (GFP) under the regulation of the insulin promoter. AdINSGFP was developed by de Vargas et al. (J. Biol. Chem. 272: 26573-26577 (1997). The HPDE6c7 cells were infected according as follows. HPDE6c7 cells were plated on plastic cell culture dishes and incubated in KBM for two days to form a monolayer. Then the cells were infected with about 50 plaque forming units (pfu) per cell of AdINSGFP for one hour. Afterwards, the cells were washed and maintained in KBM for three days. FIG. 8A shows a phase-contrast photomicrograph of the monolayer after three days. FIG. 8B is a fluorescence microphotograph that shows that after three days, particular cells in the monolayer expressed GFP, indicating that in those cells cell conditions are such that the insulin promoter is turned on. The results show that the insulin promoter is active in particular HPDE6c7 cells.

Nicotinamide is known to affect cell differentiation. Therefore, the effect of nicotinamide on the activity of the insulin promoter in HPDE6c7 cells was determined. The AdINGFP-infected cells were plated on plastic cell culture dishes for two days. Then the cells were infected with about 50 pfu/cell AdINSGFP for one hour. Afterwards, the cells were washed and incubated for three days in KBM containing 10 mM nicotinamide. FIG. 9A shows a phase-contrast photomicrograph of the monolayer after three days growth in KBM containing nicotinamide. FIG. 9B is a fluorescence microphotograph that shows that after three days, the insulin promoter is turned on in a substantial number of cells (FIG. 9B). The results show that nicotinamide induces a substantial number of cells to activate the insulin promoter.

RT-PCR was used to determine whether the HPDE6c7 cells are producing RNA encoding insulin. HPDE6c7 cells were incubated in either RPMI-1640 medium, complete KSF medium, KBM medium, KBM medium containing 10 mM nicotinamide, KBM medium containing 3 nM betacellulin, or KBM containing 10 mM nicotinamide or 3 nM betacellulin. Afterwards, cells grown under each of the above conditions were harvested and processed as in Example 5. RT-PCR was performed as in Example 5 except that the primers for Pdx-1 (Pdx-1 (aka IPF-1, STF-1, or IDX-1) is a homeodomain protein that is expressed at the earliest stage the dorsal and ventral foregut epithelial cells become committed towards a pancreatic destiny) were 5′-GATAAGAAACGTAGTAGCGGG-3′ (SEQ ID NO:5) and 5′-CGACGTGGCGCGACGCTGGAG-3′ (SE ID NO:6).

FIG. 10 shows that the HPDE6c7 cells express RNA encoding insulin. Lane 1 is the molecular weight markers. Lanes 2 through 7 show the RT-PCR product using primers for Pdx-1, lanes 8 through 13 show the RT-PCR product using primers for insulin, and lanes 14 through 19 show the RT-PCR product using primers for beta-actin. Lanes 2, 8, and 14 show the RT-PCR product for HPDE6c7 cells incubated in RPMI-1640 medium; lanes 3, 9, and 15 show the RT-PCR product for HPDE6c7 cells incubated in complete KSF medium; lanes 4, 10, and 16 show the RT-PCR product for HPDE6c7 cells incubated in KBM medium; lanes 5, 11, and 17 show the RT-PCR product for HPDE6c7 cells incubated in KBM medium containing 10 mM nicotinamide; lanes 6, 12, and 18 show the RT-PCR product for HPDE6c7 cells incubated in KBM media containing betacellulin; and, lanes 7, 13, and 19 show the RT-PCR product for HPDE6c7 cells incubated in KBM medium containing 10 mM nicotinamide and betacellulin. Thus, the cells are capable of differentiating into insulin-producing cells.

### EXAMPLE 8

This example is to characterize the effect that mitogens, differentiation agents, and extracellular matrix have on proliferation and differentiation of HPDE6c7 and HPDE-11 cells, in vitro.

The two major forms of diabetes, insulin-dependent diabetes (IDDM) and non-insulin dependent diabetes, are manifested as a reduction in the delivery of insulin required to maintain glucose homeostasis. Advances have been made in understanding the mechanisms for diminished insulin delivery in both IDDM and NIDDM. A major therapeutic goal in the treatment of diabetes is to re-establish metabolically regulated insulin secretion such that the timing of insulin delivery is tightly coordinated with the plasma glucose levels of the diabetic patient. Successes in both segmental pancreas and pancreatic islet transplantation have indicated that replacement of pancreatic beta cells can serve as a means for achieving metabolically-regulated insulin delivery. However, this approach has been hampered because donor pancreatic tissue is limited and because it is difficult and expensive to isolate large quantities of pancreatic islets. Thus, other sources of transplantable beta cells need to be developed for pancreatic beta cell replacement therapies. One potentially renewable source of human pancreatic beta cells could be derived from precursor cells associated with the pancreatic ductal epithelium.

The pancreas develops as a dorsal and ventral envagination of the foregut epithelium into the surrounding splanchnic mesoderm. At the earliest stage of commitment towards pancreatic fate, the dorsal and ventral foregut epithelial cells express the homeodomain protein Pdx-1 (Ohlsson et al., EMBO J. 12: 4251-4259 (1993); Ahlgren et al., Development 122: 1409-1416 (1996)) (also know as IPF-1, STF-1, and IDX-1). After a complex epithelial-mesenchymal interaction, which occurs during the development of many tissues, there is branching of the epithelium into primitive duct structures and this is followed by differentiation of exocrine and endocrine cells (recently reviewed in Edlund, Diabetes 47: 1817-1823 (1998)). At this stage of pancreas development, many investigators believe that primordial islets of Langerhans are formed from pluripotent endocrine cells budding from ductal cells (reviewed in Githens, Development and Differentiation of Pancreatic Duct Epithelium. Biliary and Pancreatic Ductal Epithelia: pathobiology and Pathophysiology. Sirica and Longnecker (Eds.). Marcel Dekker, Inc. New York (1997), pp. 323-348); Vinik et al., Horm. Metab. Res. 29: 278-293 (1997)).

Recent studies by Bouwens and colleagues have provided direct evidence that during fetal and neonatal islet formation, ductal epithelial-like precursor cells can differentiate into islet endocrine cells (Bouwens et al., Diabetes 43: 1279-1283 (1994); Bouwens et al., J. Histochem. Cytochem. 44: 947-951 (1996); Bouwens et al., Diabetologia 40: 398-404 (1997)). For these studies, cytokeratin expression was used as epithelial cell lineage markers to follow the ontogeny of islet cells. In the adult pancreas, cytokeratins 8 and 18 are expressed in exocrine acinar, duct cells, and endocrine cells, whereas cytokeratins 7, 19 and 20 are normally restricted to only ductal epithelial cells (Bouwens et al., J. Pathol. 184: 234-239 (1998)). Using cytokeratin 20 as an ductal epithelial marker, it was found that all epithelial cells within the rat pancreatic rudiment at gestational day 13 expressed cytokeratin 20 (Bouwens et al., J. Histochem. Cytochem. 44: 947-951 (1996)). Between day 17 and birth, large aggregates of ductal cells expressing cytokeratin 20 were formed and these gradually developed into endocrine cells (Bouwens et al., J. Histochem. Cytochem. 44: 947-951 (1996)). Vimentin and bcl-1 also have a similar pattern of expression as observed with cytokeratin 20, suggesting that they are markers of pancreatic ductal epithelial stem cells (Bouwens et al., J. Histochem. Cytochem. 44: 947-951 (1996)). Shortly after birth, neonatal islets were surrounded by a proliferative mantle of cytokeratin 20 expressing duct cells and as the islets matured cytokeratin 20 expression within the mantle diminished (Bouwens et al., Diabetes 43: 1279-1283 (1994)). These studies strongly demonstrate that islet morphogenesis can occur from pluripotent duct epithelial cell aggregates. Recapitulation of these processes in vitro may provide a means for generating pancreatic beta cells.

Pancreatic duct cells retain the capacity to differentiate into endocrine cells after the postnatal period. Islet neogenesis from ducts have been shown in a variety of rodent models including; alloxan-induced diabetic rabbits or mice (Bencosme, Am. J. Pathol. 31: 1149-1164 (1955); Patent et al., Acta Anat. (Basel) 66: 504-519 (1967)), 90% pancreatectomized adult rats (Brockenbrough et al., Diabetes 37: 232-236 (1988); Bonner-Weir et al., Diabetes 42: 1715-1720 (1993); Sharma et al., Diabetes 48: 507-513 (1999)), cellophane-wrapped pancreas of adult hamsters (reviewed in Vinik et al., Horm. Metab. Res. 29: 278-293 (1997); Rosenberg, Cell Transplant. 4: 371-383 (1995)), transgenic mice expressing a variety of cytokines or growth factors (Sarvetnick et al., Adv. Exp. Med. Biol. 321: 85-89 (1992); Gu et al., Development 118: 33-46 (1993); Wang et al., J. Clin. Invest. 92: 1349-1356 (1993); Wang et al., Diabetologia 38: 1405-1411 (1995)) and pancreatic duct-ligated rats (Wang et al., Diabetologia 38: 1405-1411 (1995)). The nature of the pluripotent cells involved in neogenesis of islet cells has been controversial, however, recent evidence suggests that there are transitional stages of differentiation between ductal and islet cells. Thus, Wang et al. (Diabetologia 38: 1405-1411 (1995)) have shown that cytokeratin 20 and insulin or glucagon co-localize during islet neogenesis in pancreatic duct-ligated rats, suggesting a transition directly from ductal epithelial cells into islet cells. Recent studies in 90% pancreatectomized rats, have shown that ductal cell proliferation precedes the appearance of Pdx-1 in daughter cells (Sharma et al., Diabetes 48: 507-513 (1999)). Pdx-1 is not normally expressed in adult pancreatic ductal epithelial cells, but is expressed at high levels in mature beta cells (Ohlsson et al., EMBO J. 12: 4251-4259 (1993); Guz et al., Development 121: 11-18 (1995)). Overall these data suggest that ductal cells can transiently regain pluripotency and differentiate towards endocrine cell phenotypes.

The existence of a pluripotent ductal epithelial cells that can differentiate into endocrine cells, raises the possibility that isolation and in vitro culture of these cells may be used to generate pancreatic beta cells. Recently, we have been characterizing two human pancreatic ductal epithelial cell lines (HPDE6c7 and HPDE-11 cells) derived from normal primary human pancreatic ductal epithelial cells (Furukawa et al., Amer. J. Path. 148: 1763-1770 (1996)). The HPDE cells were transformed by retrovirus-mediated expression of the E6 and E7 genes of the human papilloma virus 16. Previous studies have shown that HPDE cells are positive for human cytokeratins 8, 18, and 19 (Furukawa et al., Amer. J. Path. 148: 1763-1770 (1996)). In addition, we have shown that the HPDE cells express human cytokeratin 7, suggesting that these cells are truly derived from pancreatic ductal epithelium. The HPDE cells also express vimentin and bcl-2, which are putative markers of pancreatic epithelial stem cells (Bouwens et al., J. Histochem. Cytochem. 44: 947-951 (1996). When HPDE cells are cultured on MATRIGEL they develop a ductule-like structure and express ductal cell markers. Remarkably, when HPDE cells are cultured in nicotinamide and/or hepatocyte growth factor the cells appear to synthesize and release insulin.

Overall, our preliminary studies on HPDE cells suggest that they are not terminally differentiated and have the capacity to differentiate into pancreatic ductal cells or endocrine cells. We hypothesize that the correct combination of mitogens, differentiation agents, and extracellular matrix will allow us to direct the HPDE cells towards an endocrine cell phenotype. If this is possible the HPDE cell lines are predicted to serve as useful models needed to understand the molecular processes involved in differentiation of human pancreatic ductal epithelial cells to beta cells.

HPDE6c7 cells are human pancreatic ductal epithelial cells transformed by expression of the E6 and E7 genes from human papilloma virus, which have the ability to differentiate into ductal epithelium cells or insulin-producing cells. The HPDE6c7 cells are a good in vitro model system with which to study the differentiation processes involved in generation of human islet cells from pancreatic ductal epithelial cells. The correct combination of mitogens, differentiation agents, and extracellular matrix will enable directing the HPDE6c7 cells towards a differentiated beta cell phenotype. However, the HPDE6c7 cells need to be further characterized. Therefore, the growth and differentiation of the cells when cultured on standard cell culture dishes are compared to growth on MATRIGEL (growth factor reduced). The effects of culturing the HPDE6c7 cells with agents previously described to increase beta cell differentiation or proliferation are examined. The agents to be examined include, but are not limited to nicotinamide, sodium butyrate, activin A, betacellulin, prolactin, placental lactogen, growth hormone (GH), insulin like growth factors (IGF-1 and -2), hepatocyte growth factor (HGF), vascular endothelial growth factor (VEGF), basic fibroblast growth factor (bFGF), epithelial growth factor (EGF), transforming growth factor-alpha (TGF-α) and gastrin.

Several mitogens and differentiation agents have been shown to be associated with the regulation of beta cell differentiation, replication, and maintenance of beta cell mass (recently reviewed in Vinik et al., Horm. Metab. Res. 29: 278-293 (1997); Vinik et al., Diabetes Rev. 4: 235-263 (1996); Bonner-Weir et al., Trends Endocrinol. Metab. 5: 60-64 (1994)). In this example, the effect of growth factors including IGF-1, GH, betacellulin, and prolactin are examined because they have been described to be mitogenic for beta cells and insulinoma cell lines (Brelje et al., Diabetes 43: 263-273 (1994); Huotari et al., Endocrinol. 139: 1494-1499 (1994)). IGF-1 is examined because it has been implicated in islet neogenesis from ductule epithelium after partial pancreatectomy in rats (Bonner-Weir et al., Recent Prog. Horm. Res. 49: 91-104 (1994)). HGF is examined because its receptor (c-met) has been shown to be expressed at high levels in human pancreatic ductal epithelium (Vila et al., Lab. Invest. 73: 409-418 (1995)) and beta cells (Otonkoski et al., Endocrinol. 137: 3131-3139 (1996)). HGF also has been shown to increase proliferation of fetal human beta cells (Otonkoski et al., Endocrinol. 137: 3131-3139 (1996)) and pancreatic duct cells (Vila et al., Lab. Invest. 73: 409-418 (1995)). In addition, our preliminary data has shown that incubation of HPDE6c7 cells with HGF and nicotinamide increases the appearance of insulin in the media. VEGF is examined because its receptors are found on ductal epithelial cells (Oberg et al., Growth Factors 10: 115-126 (1994)) and VEGF stimulates ductal cell proliferation (Oberg et al., Mol. Cell. Endocrinol. 126: 125-132 (1997)). TGF-α is examined because it is abundantly expressed in the developing pancreas (Miettinen et al., Development 114: 833-840 (1992)). In addition, overexpression of TGF-α and gastrin in transgenic mice has been reported to significantly increase beta cell mass from ductal precursor cells (Wang et al., J. Clin. Invest. 92: 1349-1356 (1993)). EGF is examined because the EGF receptor is expressed throughout the fetal pancreas and mice lacking a functional EGF receptor have impaired epithelial development in several organs including the pancreas (Miettinen et al., Nature (Lond.) 376: 337-341 (1995); Miettinen et al., Diabetologia (Suppl 1) 40: A25 (1997)). Betacellulin and activin A are examined because combinations of these factors have been shown to convert AR42J cells (a rat pancreatic acinar cell line) to insulin expressing cells (Mashima et al., J. Clin. Invest. 97: 1647-1654 (1996); Mashima et al., Diabetes 48: 304-309 (1999)). Furthermore, betacellulin was shown to be required for insulin and glucokinase gene expression when α-TC1 cells transfected with the Pdx-1 gene (Watada et al., Diabetes 45: 1826-1831 (1996)).

Additional differentiation agents that are examined include nicotinamide and sodium butyrate. Nicotinamide has been shown to increase differentiation of fetal human pancreatic islet cells (Otonkoski et al., J. Clin. Invest. 92: 1459-1466 (1993)) and increases islet neogenesis in animals after partial pancreatectomy (Yonemura et al., Diabetes 33: 401-404 (1984)). In addition, our preliminary data suggests that nicotinamide differentiates the HPDE cells towards an insulin-producing phenotype (see Preliminary Studies). Sodium Butyrate has been shown to increase differentiation of both RIN cells (Philippe et al., Mol. Cell Biol. 7: 560-563 (1987)) and INS-1 cells (Houtari et al., Endocrinol. 139: 1494-1499 (1994)).

### Experimental Plan

Our preliminary results have indicated that HPDE6c7 cells are pluripotent, with the capacity to differentiate into ductal epithelium cells or insulin-producing cells. Therefore, the correct combination of mitogens, differentiation agents, and extracellular matrix will allow directing the HPDE cells towards a differentiated beta cell phenotype. To test this, the HPDE6c7 cells are cultured on standard tissue culture dishes or on MATRIGEL (Collaborative Biomedical Products, Bedford, Mass.) in KBM or KSFM with epidermal growth factor and bovine pituitary extract. The cells are incubated for 24 hrs to 7 days with or without the various mitogens and differentiation agents. Many mitogens and differentiation agents are available from commercial sources; however, others are not. Recombinant human betacellulin and activin A are available from Dr. Masaharu Seno (Okayama University, Japan). Proper controls are performed for all experiments. Initial concentrations for the various mitogens and differentiation agents are determined from the literature.

For a particular agent that causes a change in differentiation, appropriate dose response studies are performed. For many of the experiments, a combination of mitogens and differentiation agents are examined. This is important because there have been many reports that suggest that only a combination of mitogens and differentiation agents results in generation of insulin-producing phenotypes. For example, the conversion of AR42J cells from a acinar cell phenotype to an insulin-producing phenotype occurred only with a combination of betacellulin and activin A (Mashima et al., J. Clin. Invest. 97: 1647-1654 (1996); Mashima et al., Diabetes 48: 304-309 (1999)). After mitogen- or differentiation agent-treatment, or both, cells are harvested and the expression levels of a variety of pancreatic beta cell- and ductal cell-gene products are determined. In addition, media insulin and C-peptide levels, and insulin contents are determined. Cells are harvested from MATRIGEL using MATRISPERSE (Collaborative Biomedical Products, Bedford, Mass.). Cell proliferation studies are performed as described below.

### A. Insulin and C-Peptide Levels

Insulin released into the media is determined by a human-specific insulin radioimmunoassay (RIA, Linco, St. Charles, Mo.). C-peptide levels is determined by use of a human-specific C-peptide RIA (Linco, St. Charles, Mo.). Measurements of C-peptide are important because it will indicate whether insulin is be properly processed before secretion. Insulin and C-peptide release is normalized to cellular DNA concentration. Cellular DNA is measured by fluorometry. Insulin content is determined by RIA after acid-ethanol extraction (Santerre et al., Proc. Natl. Acad. Sci. USA 78: 4339-4343 (1981)).

### B. Fluorescence and Confocal Microscopy

We propose that the HPDE6c7 cells have the potential to differentiate into endocrine producing cell types under the appropriate growth conditions. As described under preliminary studies section, when the HPDE6c7 cells were cultured on MATRIGEL they rapidly organized into a network of tubular/ductal structures with extensive budding. This suggested that HPDE6c7 cells when cultured on MATRIGEL may be stimulated to differentiate into insulin- and other hormone-producing cells. We do not anticipate that all of the cells in the tubular structure will have an insulin producing phenotype. At this point, we believe that it is the cells budding from the tubular structure that have the capacity to differentiate into endocrine cells. Therefore, immunofluorescence microscopy is used to localize which cells have a beta cell-like phenotype. The tubular structures formed by growing the HPDE6c7 cells on MATRIGEL are mechanically teased from the MATRIGEL or isolated from the MATRIGEL using MATRISPERSE. The cell structures are then mounted onto poly-L-lysine- or 3-aminopropyltriethoxy silane-treated slides. The cells are fixed, usually with a mix of methanol and acetic acid, and blocked. Following blocking, the cells are incubated at room temperature with primary antibodies diluted in the blocking buffer. After washing, the cells are incubated at room temperature with fluorescent-labeled secondary antibodies. Afterwards, the cells are embedded in a glycerol-based mounting medium and examined for immunofluorescence using the Ultima Laser Cytometer (Meridian Instruments, Okemos, Mich.) equipped with a confocal microscope. Confocal microscopy allows identification of which cells within the tubular structures have a beta cell phenotype. In addition, we are able determine the spatial distribution of the fluorescence in the different regions of the cells.

### C. Western Analysis

Protein levels are determined by Western analysis using specific antiserum. In short, cellular extracts (30 μg) are resolved on 10% SDS-polyacrylamide gels and electrotransferred onto Immobilon PVDF membranes. Immunoreactive proteins are detected by use of specific antiserum. Membranes are then be probed with secondary antibodies conjugated with horseradish peroxidase and visualized by chemiluminescence (Super signal substrate kit, Pierce, Rockford, Ill.). The Western blots are then stripped and re-probed with beta-actin-specific antiserum (Sigma, St. Louis, Mo.) to insure equal loading. Western blots are quantified using an Arcus II scanning densitometer (AGFA-Gavaert, N.V., Belgium) and NIH image software.

### D. Determination of mRNA Levels

Messenger RNA levels are examined. The mRNA levels examined include, but are not limited to, mRNAs for insulin, glucokinase, GLUT 2, sulfonylurea receptor, Pdx-1, BETA2, glucagon, somatostatin, CFTR, carbonic anhydrase and cytokeratins. Expression of mRNA levels are determined by Northern analysis using various cDNAs as a hybridization probes. In short, total RNA is isolated using an acid phenol method (Chomczynski and N. Sacchi, Anal. Biochem. 162: 156-159 (1987)). Total RNA is fractionated on a 1.5% agarose-formaldehyde gel and transferred to a nylon membrane by capillary blotting. Membranes are UV cross-linked and pre-hybridized as previously described (Olson et al., Proc. Natl. Acad. Sci. 92: 9127-9131 (1995)). Membranes are hybridized with ³²P-labeled cDNA probes. Probes are labeled with ³²P-dCTP by use of a random primers method (Feinberg and Vogelstein, Anal. Biochem. 137: 266-267 (1984)). Hybridization is assessed by autoradiography and quantified on a Molecular Dynamics phosphoimager (Storm 820). Blots are then be stripped and rehybridized with a ³²P-labeled DNA probe for β-actin mRNA to control for loading.

Alternatively, mRNA levels will be measured using a competitive RT-PCR protocol as described by Gilliland et al. (Proc. Natl. Acad. Sci. USA 87: 2725-2729 (1990)) and as modified by Iwashima et al. (Diabetes 42: 948-955 (1993)). In short, cDNA is synthesized using equivalent amounts (i.e., 0.5 μg) of total RNA in a 20 μl reaction containing 50 mM Tris HCl (pH 8.3), 75 mM KCl, 3 mM MgCl₂, 10 mM DTT, 0.5 mM of each dNTP, 2U RNasin, 100 pmol pd(N6) random hexamers (Pharmacia Biotechnology), and 200U Moloney murine leukemia virus reverse transcriptase. PCR primers are designed to target specific sequences within the mRNAs. Included in the PCR reactions are internal standard cDNAs designed to contain a unique cleavage site for a restriction enzyme so that PCR amplification the internal standard cDNA can be distinguished from the endogenous target cDNAs. PCR is performed in 50 μl PCR buffer (Perkin Elmer Cetus) containing 0.2 μM sense and anti-sense primers, 200 μW dNTP, 1 μCi [α-³²P]dCTP (3000 Ci/mmol), 5U Taq polymerase and increasing concentrations (varying from 0.1 fg to 10 pg) of internal standard cDNA. The PCR products are digested with the appropriate restriction enzymes and fractioned by electrophoresis through a 5% polyacrylamide gel. This enables endogenous targeted cDNA (mRNA) to be distinguished from the internal standard cDNA. Bands corresponding to the predicted PCR products are excised from the gel and ³²P incorporation is determined by liquid scintillation counting.

### E. HPDE Cell Proliferation

We predict that the mitogens and differentiation agents will have marked effects on HPDE cell proliferation. For experiments with HPDE cells cultured on standard tissue culture dishes, we measure cellular proliferation using an ELISA-based BrdU incorporation assay (Amersham Life Science, Inc) as described in Specific Aim 3. Although this assay gives a measure of cellular proliferation in cells grown on MATRIGEL, it does not provide any information on which cells are proliferating within the tubular structure. Therefore, for cells cultured on MATRIGEL we use a BrdU immunostaining kit (Amersham Life Science, Inc) that enables us to directly determine the location of proliferative cells within the tubule structure.

In short, cells grown on MATRIGEL are labeled for 1 hr with BrdU, the ductule structure is isolated, mounted on slides and fixed in acid-ethanol. BrdU incorporation is detected by immunostaining according to manufacturer's specifications. At this time, we predict that the mitogens will cause increased proliferation of the tubule structures and this will be mainly localized to the buds.

### Specific Aim 2

Examine whether transplantation of HPDE cells into different anatomical sites within alloxan-induced diabetic athymic mice have effects on proliferation and differentiation of HPDE cell.

We hypothesize that the correct combination of extracellular matrix and growth factors can drive HPDE6c7 cells towards differentiated pancreatic ductal or endocrine cells. In a variety of animals models, regeneration of pancreatic beta cells can be derived from cells associated with ductule epithelial cells. For example, in 90% pancreatectomized rats there is regeneration of both acinar and endocrine tissue (Brockenbrough et al., Diabetes 37: 232-236 (1988)). The increase in beta cell mass in this model is due to hypertrophy and hyperplasia of existing beta cells and regeneration of beta cells from ductal epithelium (Brockenbrough et al., Diabetes 37: 232-236 (1988)). Similarly, following alloxan-induced diabetes there is regeneration of insulin-containing cells from small pancreatic ductules (Bencosme, Am. J. Pathol. 31: 1149-1164 (1955); Hughes, J. Anat. 81: 82 (1947)). These studies suggest that marked decreases in beta cell mass can lead to regeneration of beta cells derived from the ductule epithelium and suggests that signals for beta cell regeneration are generated within the pancreatic environment. We hypothesize that the signals involved in beta cell regeneration are able to direct HPDE6c7 cells towards pancreatic endocrine cell differentiation. Therefore, transplantation of HPDE6c7 cells into animals with reduced beta cell mass is predicted to be sufficient to direct HPDE6c7 cell differentiation. To test this hypothesis, HPDE6c7 cells are transplanted into two anatomical environments, the pancreas and under the kidney capsule, in either alloxan- or streptozotocin-induced diabetic athymic (nude) mice. The state of differentiation of transplanted HPDE6c7 cells is determined by immunohistochemical analysis for a selected group of beta cell-specific genes and pancreatic ductal cell genes.

### Experimental Plan

The effect that decreased beta cell mass has on HPDE6c7 cell line differentiation in vivo is studied by transplanting HPDE6c7 cells into alloxan- or streptozotocin-induced diabetic athymic mice. The athymic mice are housed in a sterile isolation facility with free access to sterile laboratory chow and water. The initial transplantation studies are focused on alloxan-induced diabetic athymic mice. We have chosen alloxan-induced diabetes over streptozotocin-induced diabetes, because some investigators have found that there is little beta cell regeneration when diabetes is induced in adult rats with streptozotocin (Komai, Acta Histochem. Cytochem. 14: 261 (1981); Morohoshi et al., Acta Pathol. Jpn. 34: 271-281 (1984); Michels et al., Proc. Soc. Exp. Biol. Med. 184: 218-224 (1987)). In contrast, when alloxan is used to induce diabetes in adult rats there is regeneration of insulin containing cells from the ductal epithelium (Bencosme, Am. J. Pathol. 31: 1149-1164 (1955); Hughes, J. Anat. 81: 82 (1947)).

Male Balb C NU/NU mice, 5 to 7 weeks of age, are made diabetic by a single intravenous injection of alloxan (Sigma, St. Louis, Mo., 90 mg/kg body weight) as described by Korsgren et al. (Surgery 113: 205-214 (1993)). Before transplantation of HPDE6c7 cells, diabetes is confirmed by the presence of blood glucose levels above 20 mM, weight loss, polydipsia, and polyuria. Blood glucose levels are taken in non-fasting conditions. Blood collection are done from the hind leg vein and glucose levels assessed using a hand held glucose monitor (Boehringer-Mannheim).

Five groups of animals are tested: 1) alloxan-induced diabetic mice undergoing a mock transplantation (receiving no HPDE6c7 cells); 2) alloxan-induced diabetic mice transplanted with HPDE6c7 cells under the kidney capsule; 3) alloxan-induced diabetic mice transplanted with HPDE6c7 cells into the pancreas; 4) control mice transplanted with HPDE6c7 cells under the kidney capsule; 5) control mice transplanted with HPDE6c7 cells into the pancreas. preferably, there are 6 mice per group. Time points are examined at two, four, and eight weeks post-transplantation of HPDE6c7 cells into the athymic mice. We are aware that hypoglycemia may limit the length of time for which these studies can be carried out.

As a starting point, we are planning to transplant 10⁶ HPDE6c7 cells into the pancreas or under the kidney capsule of the alloxan-induced diabetic mice. Initial studies, however, are performed to determine how many HPDE6c7 cells need to be transplanted so that changes in differentiation can be assessed. HPDE6c7 cells are transplanted as cell aggregates, because previous studies have shown that transplantation of fetal islet cells as cell aggregates increases development of mature islet-like structures (Beattie et al., Diabetes 45: 1223-1228 (1996) and personal communication with Dr. Bonner-Weir, Joslin Diabetes Center, Boston, Mass.). HPDE6c7 cell aggregates are generated by mild trypsinization of monolayers of HPDE6c7 cells. Alternatively, cell aggregates are generated by mild trypsinization of HPDE6c7 ductule-like structures isolated from cells cultured on MATRIGEL. In addition, HPDE6c7 cell aggregates are pretreated with nicotinamide before transplantation. Pretreatment of HPDE6c7 cells aggregates is done because studies have shown that pretreatment of human or porcine islet-like cell clusters (aggregates) with nicotinamide increases differentiation and function of cells when transplanted into nude mice (Korsgren et al., Surgery 113: 205-214 (1993); Beattie et al., Diabetes 45: 1223-1228 (1996)).

HPDE6c7 cells can be fluorescent tagged so that they can be easily tracked after transplantation. HPDE cells are tagged by loading cells with fluorescent lipophilic tracers such as DiI or DiO (Molecular Probes, Eugene, Oreg.). These fluorescent probes easily incorporate into plasma membranes and exhibit very low cell toxicity. The advantage of these fluorescent probes is that they remain incorporated into the plasma membranes even after multiple rounds of cell division. DiI and DiO can be used with standard fluorescein and rhodamine optical filters, respectively. Importantly, some of the newer DiI analogs are stable after tissue fixation. If tagging proves insufficient, in situ hybridization is used to identify expression of the neomycin-resistance gene (a marker of the HPDE6c7 cells) and then use in situ hybridization on adjacent sections to measure the expression of beta cell specific genes.

HPDE cell aggregates are transplanted under the kidney capsule as described for porcine and rat pancreatic islets by Davalli et al. (Diabetes 44: 104-111 (1995)). In short, HPDE cells are mildly trypsinized, stained with DiI or DiO, and washed multiple times with sterile PBS. Cell aggregates are aspirated into a 200 μl pipette tip and allowed to settle by gravity. Cells are then transferred to a polyethylene tube (PE-50, Becton Dickinson, Parsippany, N.J.) via a Hamilton syringe. The polyethylene tube is then bent and centrifuged at 400×g to pellet the cells. With the mouse under light anesthesia (Metafane), the kidney is exposed by lumbar incision. A capsulotomy is then performed in the lower pole of the kidney. The tip of the tubing is then advanced under the capsule to the upper pole of the kidney where the HPDE6c7 cells are injected with the Hamilton syringe. The capsulotomy is then cauterized.

HPDE6c7 cell aggregates are also transplanted directly into the pancreas as described for neonatal islets by Hayek and Beattie (Metabolism 41: 1367-1369 (1992)). In short, HPDE cells are mildly trypsinized, stained with DiO or DiI, washed, and transferred to a 25 gauge butterfly infusion set. Following metafane anesthesia, a midline or lateral abdominal incision is made and cells are directly injected into the pancreatic parenchyma.

After transplantation, blood glucose concentrations and body weight are monitored weekly. Blood glucose levels are determined between 8:00 and 10:00 AM using a hand-held glucose meter (Boehringer-Mannheim). Improvements in blood glucose concentrations over time provide an early indication whether the transplanted HPDE6c7 cells have differentiated and are producing insulin. The source of insulin could be from either regenerating endogenous beta cells or from the transplanted beta cells. However, the source of insulin can be inferred by comparing mice receiving transplanted HPDE6c7 cells versus mock transplanted mice.

If an improvement in blood glucose levels occurs post-transplantation of HPDE6c7 cells, then oral glucose tolerance tests (OGTTs) or intraperitoneal glucose tolerance tests (IGTTs) are performed before the mice are sacrificed. Either OGTTs or IGTTs are performed after a 2 hr food deprivation. For OGTTs, 2 g/kg D-glucose is infused endogastrically through a PE-50 polyethylene tube. For IGTTs, 2 g/kg of D-glucose is injected intraperitoneally. Blood samples are then collected from a snipped tail 0, 5, 15, 30, 60, 90, and 120 minutes after glucose administration. Blood glucose levels are determined using a hand-held glucose meter (Boehringer-Mannheim).

Two, four, and eight weeks after transplantation, the mice are sacrificed and the differentiation state of the transplanted cells is determined. At the time of sacrifice, a large blood sample is collected by cardiac puncture. Serum glucose, insulin, and C-peptide levels are determined from these samples. Serum glucose levels are determined using a dual glucose and lactate analyzer (YSI incorporated, Yellow Springs, Ohio). Human insulin levels and human C-peptide levels are determined using human-specific RIA kits from Linco Research Inc (St. Charles, Mo.). Endogenous insulin secretion is assayed using a rat-specific C-peptide RIA kit (Linco Research Inc, St. Charles, Mo.) that is 100% cross reactive to mouse C-peptide but does not cross react with human C-peptide. Comparison of the results generated from the rat- and human-specific RIAs enables a determination as to whether the circulating insulin and C-peptide come from HPDE6c7 cells or from regenerated endogenous mouse beta cells.

Analysis of HPDE cell differentiation is primarily determined by immunohistochemistry. Expression of insulin, glucagon, somatostatin, GLUT 2 and Pdx-1 is assessed. In addition, the expression of at least two ductal cell proteins including carbonic anhydrase and CFTR, and a variety of cytokeratins, are determined. In short, tissue at the site of HPDE6c7 cell transplantation is excised and formalin fixed. The fixed tissue is then paraffin embedded and sectioned. The sections are then mounted on slides and deparaffinized. Depending on the antigen and antibodies, a variety of antigen retrieval steps are performed. Sections are then probed with the various primary antibodies. Immunoreactivity is then detected using secondary antibodies conjugated with horseradish peroxidase, FITC, or rhodamine. If the immunoreactivity signals are weak, a fluorescent-avidin kit from Vectors Laboratories Inc (Burlingame, Calif.) to amplify the signals is used. This kit uses biotinylated-secondary antibodies and Immunoreactivity signals are amplified by use of fluorescent labeled-avidin biotin complexes. To assure that the transplanted cells are the source of immunoreactivity, DiI or DiO fluorescence in adjacent sections is visualized.

If transplantation of HPDE6c7 cells into diabetic animals results in differentiation of HPDE6c7 cells towards a beta cell phenotype, the cells are excised and propagated in cell culture. This enables a determination as to whether the change in the HPDE6c7 cell phenotype is stable. When excised cells are transferred to cell culture, they may be contaminated with mouse fibroblasts. Since HPDE cells are neomycin-resistant, contaminating mouse cells are removed by culturing cells in media supplemented with 400 μg/ml neomycin. Once HPDE6c7 cells are re-established in cell culture, the expression of pancreatic beta cell genes is examined to determine whether the cells secrete insulin in response to glucose and other secretagogues.

In addition to increasing differentiation, transplantation of HPDE cell into alloxan-treated mice may lead to enhanced cell growth. Differences in HPDE cell proliferation after transplantation is measured by monitoring BrdU incorporation into DNA of transplanted cells. This is done as previously described by Davilli et al. (Diabetes 44: 104-111 (1995)). In short, six hours before the animals are to be sacrificed the mice are injected with 100 mg/kg BrdU. As described previously, BrdU is a thymidine analog and is incorporated into newly synthesized DNA. Six hrs later mice are sacrificed and transplanted cells are isolated. The recovered cells are first fixed in Bouin's solution overnight and then fixed in 10% formalin. The cells are then embedded in Araldite and sectioned. The cell sections are then stained for BrdU using an anti-BrdU antibody (Amersham Life Science Inc) as previously described by Montana et al. (J. Clinical Investigation 91: 780-787 (1993)).

### Specific Aim 3

Examine whether expression of transcription factors involved in beta cell development and maintenance can regulate HPDE cell proliferation and differentiation.

The major goal is to determine whether HPDE6c7 cells have the capacity to differentiated towards pancreatic beta cells. Recently, a number of transcription factors (Pdx-1, Isl-1, Pax 4, Pax 6, BETA2, Nkx2.2) have been identified that function at different stages during pancreas development (Jonsson et al., Nature 371: 606-609 (1994); Offield et al., Development 122: 983-995 (1996); Ahlgren et al., Nature 385: 257-260 (1997); Sosa-Pineda et al., Nature 386: 399-402 (1997); St-Onge et al ., Nature 387: 406-409 (1997); Naya et al., Gene Dev. 11: 2323-2334 (1997); Sussel et al., Development 125: 2213-221 (1998)). We hypothesize that controlled expression of some of these transcription factors may be sufficient to direct HPDE cells towards a beta cell phenotype. One of the primary transcription factors that is tested is Pdx-1 (also termed IPF1, STF-1, IDX-1). Pdx-1 is a homeodomain transcription factor that is expressed early (mouse embryonic day 8.5) during the development of the pancreas (Ohlsson et al., EMBO J. 12: 4251-4259 (1993); Leonard et al., Mol. Endo. 7: 1275-1283 (1993); Miller et al., EMBO J. 13: 1145-1156 (1994)). Studies have shown that mice containing a targeted disruption of the pdx-1 gene are born apancreatic (Jonsson et al., Nature 371: 606-609 (1994); Offield et al., Development 122: 983-995 (1996)), emphasizing the importance of Pdx-1 in pancreatic development. In the adult pancreas, Pdx-1 expression is restricted to the pancreatic beta cells residing in the islets of Langerhans (Ohlsson et al., EMBO J. 12: 4251-4259 (1993); Guz et al., Development 121: 11-18 (1995)). Pdx-1 has been proposed to regulate the expression of a variety of pancreatic endocrine cell genes, including insulin (Ohlsson et al., EMBO J. 12: 4251-4259 (1993); Peshavaria et al., Mol. Endo. 8: 806-816 (1994); Serup et al., Biochem. J. 310: 997-1003 (1995); Peers et al., Molecular Endocrinology 8: 1798-1806)), somatostatin (Leonard et al., Mol. Endo. 7: 1275-1283 (1993); Miller et al., EMBO J. 13: 1145-1156 (1994)), GLUT2 (Waeber et al., Mol. Endocrinol. 10: 1327-1334 (1996)), islet amyloid polypeptide (Watada et al., Biochem. Biophys. Res. Commun. 229: 746-751 (1996)), and glucokinase (Watada et al., Diabetes 45: 1478-1488 (1996)), through its ability to interact at A-T rich regions contained within the promoter of these genes. A recent study by Ahlgren et al. has shown that selective disruption of Pdx-1 in pancreatic beta cells markedly alters beta-cell phenotype by decreasing insulin, IAPP, and GLUT2 expression while increasing glucagon expression (Ahlgren et al., Genes & Development 12: 1763-1768 (1998)), thus providing evidence that a Pdx-1 plays pivotal role in maintenance of the beta cell phenotype. Importantly, increased levels of Pdx-1 is associated with differentiation of ductal cells to beta cells after 90% pancreatectomy in rats (Sharma et al., Diabetes 48: 507-513 (1999)). Because of the role of Pdx-1 in pancreas development, maintenance of beta cell phenotype and the regeneration of beta cells from ductal epithelial cells, it is reasonable to hypothesize that expression of Pdx-1 in HPDE cells may be capable of differentiating these cells into insulin-producing cells.

Expression of other transcription factors, alone or in combination with Pdx-1, may also be sufficient to determine the differentiation state of HPDE6c7 cells. It is beyond the scope of this proposal to extensively review the role of all these factors in the development and maintenance of pancreatic beta cells, however, a short explanation is provide as to why we may have to test these additional transcription factors. First, Pdx-1, Pax4, BETA2, Nkx2.2 and Nkx6.1 are expressed in both progenitor cells and in differentiated endocrine cells (Edlund, Diabetes 47: 1817-1823 (1998)). Mice lacking a functional pax4 gene do not develop differentiated beta cells or delta cells (Sosa-Pineda et al., Nature 386: 399-402 (1997)), while mice lacking a functional Nkx2.2 gene have reduced insulin-producing cells, alpha cells and PP cells (Sussel et al., Development 125: 2213-221 (1998)). These data suggest that expression of Pax4 or Nkx2.2 may be required for generation of beta cells. Expression of Pax4 may not be a problem, however, because some studies have suggested that Pax4 may function downstream of Pdx-1 (Edlund, Diabetes 47: 1817-1823 (1998)). In addition, Isl-1 and Pax6 are expressed in all endocrine cells (Ahlgren et al., Nature 385: 257-260 (1997); St-Onge et al., Nature 387: 406-409 (1997); Sander et al., Genes Dev. 11: 1662-1673 (1997)) suggesting that they serve an important role in the differentiation of these cells. Finally, Nkx6.1 and BETA2 are expressed in differentiated beta cells (Osteret al., J. Histochem. Cytochem. 46: 707-715 (1998)).

### Experimental Plan

We hypothesize that the correct combination of transcription factors, expressed at appropriate levels can direct HPDE6c7 cells towards a pancreatic beta cell phenotype. Eventually, this approach will allow us to generate a human pancreatic beta cell line that is appropriate for transplantation therapies. Our preliminary studies have shown that stable expression of Pdx-1 completely growth arrests HPDE6c7 cells. Interestingly, the Pdx-1-growth arrested cells are larger than control cells and appear highly granulated. The fact that Pdx-1 growth arrests HPDE6c7 cells is highly suggestive that Pdx-1 is directing these cells towards an end stage of differentiation. Because overexpression of Pdx-1 in HPDE6c7 cells limits cell proliferation, we need to employ techniques that will allow us to regulate transcription factor expression in a large number of cells. Therefore, we propose a two step approach.

Step (1). Adenovirus-mediated expression will be used to rapidly screen which transcription factors are effective at differentiating HPDE cells towards a pancreatic beta cell phenotype. Because recombinant adenoviruses are able to transduce genes into nearly 100% of the cells, we will be able to rapidly determine effects on cellular phenotype. Furthermore, adenovirus-mediated transduction of genes allows us to assess whether a combination of transcription factors are required to produce the desired phenotype.

Step (2). Once we have determined which transcription factors are effective at regulating phenotype of HPDE cells, we then make cell lines that have conditionally-regulated expression of these transcription factors. Since we have already observed that Pdx-1 overexpression limits HPDE6c7 cell proliferation and causes the cells to have a granulated appearance, we have already begun to generate conditionally-regulated Pdx-1 expressing HPDE6c7 clones.

### Step 1. Recombinant Adenovirus Expression

The first step utilizes a recombinant adenovirus-based expression system that has been successfully used to produce high levels of protein expression in both primary islets (Becker et al., In Protein expression in animal cells., M. Roth, Editor., Academic Press, Inc.: San Diego. p. 161-189 (1994); Becker et al., J. Biol. Chem. 271: 390-394 (1996)) and insulinoma cell lines (Becker et al., In Protein expression in animal cells., M. Roth, Editor., Academic Press, Inc.: San Diego. p. 161-189 (1994); Ferber et al., J. Biol. Chem. 269: 11523-11529 (1994)). This method allows us to rapidly screen which transcription factors can regulate HPDE6c7 cell phenotype. Furthermore, adenovirus-mediated expression allows us to efficiently transfer in a combination of beta cell-specific transcription factors thus allowing us to determine the combinatorial effect on HPDE6c7 cell phenotype. Our initial experiments are focused on generating recombinant adenoviruses that express Pdx-1. Rat Pdx-1 cDNA was received from Dr. Marc Montminy, Joslin Diabetes Center, Boston, Mass. The recombinant adenoviruses are prepared by inserting the transcription factor cDNA into the pACCMV.pL.pA vector adjacent to the CMV promoter. The recombinant adenoviruses is then prepared according to the method of Becker et al. (Becker et al., In Protein expression in animal cells., M. Roth, Editor., Academic Press, Inc.: San Diego. p. 161-189 (1994)).

In short, the pACCMV.pLpA-p21 plasmid containing the transcription factor inserts is allowed to recombine through homologous recombination with pJM17 in permissive human 293 cells to generate recombinant adenoviruses. Viruses are plaque purified and amplified. Insertion of transcription factor cDNAs into the recombinant adenoviruses are then confirmed by Southern analysis. High titer crude lysates of the recombinant adenoviruses are then prepared by further amplification in 293 cells.

The procedure for infecting HPDE6c7 cells with recombinant adenoviruses is as follows. HPDE cells are cultured in keratinocyte media until they have reached about 80% confluence. The cells are then cultured in media containing about 5 to 50 pfu of recombinant virus per cell for 1 hr. The cells are then rinsed with PBS and further cultured in keratinocyte media. The expression of the transcription factors is allowed to proceed for two to seven days and is confirmed by Western analysis as described below. A recombinant adenovirus (AdCMV-βGAL virus) which expresses the β-galactosidase protein is used in all of our experiments to control for non-specific viral effects and to serve as a infection efficiency marker.

### Step 2. Conditional Expression of Beta Cell-Specific transcription factors using tetracycline- or AP1510-regulated systems.

The goal of Specific Aim 3 is to determine whether controlled expression of beta cell transcription factors is capable of directing HPDE cell towards a differentiated beta cell phenotype. Ideally, this approach may generate a human pancreatic beta cell line. To finely regulate the level of transcription factor expression, we propose to generate stable HPDE6c7 cell lines in which the expression of transcription factor genes are under the control of a regulated promoter. While many inducible expression systems exist, such as heat shock-, steroid-, or metallothionein-regulated, they tend to be hindered by high basal levels of expression and pleiotropic effects that the inducing agents have on host gene expression (Yarranton, Curr. Opin. Biotechnol. 3: 506-511 (1992); Gossen et al., Trends Biochem. Sci. 18: 471-475 (1993)). Therefore, we focus on regulating transcription factor expression using tetracycline-responsive promoters, which allows very tight control of gene expression and has few pleotropic effects as originally described by Gossen and Bujard (Proc. Natl. Acad. Sci. USA 89: 5547-5551 (1992)). As an alternative method, we also use a new regulated gene expression system developed by ARIAD Pharmaceuticals, Inc.

### Method 1. Conditional Expression of Transcription Factors Involved in Beta Cell Development and Maintenance using Tetracycline-regulated Systems

The establishment of stable cell lines expressing tetracycline-inducible transcription factors utilizes a recombinant retrovirus, pRetro-On (Clontech, Palo Alto, Calif.). The pRetro-On vector was derived from the Moloney murine leukemia virus and expresses the reverse-tetracycline transactivator (the “Tet On” system) from a SV40 promoter and the puromycin resistance gene from the viral LTR, and contains a multiple cloning site downstream from the tetracycline-responsive promoter. The methods for producing high titer, helper free retroviruses using transient transfection of Phoenix cells will be those of Dr. Nolan, Stanford University, California (available over the internet at leland.stanford.edu/group/) . To produce the Retro-On-p21 virus, we clone the transcription factor coding sequences into the pRetro-On plasmid downstream from the tetracycline-responsive promoter. Next Phoenix-Ampho cells (a retroviral packaging cell line used to package infectious, yet replication incompetent viruses) are transiently transfected with the pRetro-On plasmid containing the inserts by the calcium phosphate-DNA co-precipitation method. Forty-eight hours after the transfections, pRetro-On viruses are harvested by isolating the Phoenix cell culture media and this is used directly to infect subconfluent populations of HPDE6c7 cells. Forty-eight hours after infection, infected cells are selected for puromycin resistance. Currently, we are selecting puromycin resistant cell clones from HPDE6c7 cells infected with pRetro-ON-Pdx-1. To insure that these puromycin resistant cell clones are producing the tetracycline-responsive transactivator we transiently transfect them with a tetracycline-responsive luciferase reporter gene, pUHC13-3 (Gossen and Bujard, Proc. Natl. Acad. Sci. USA 89: 5547-5551 (1992)). Cell clones resistant to puromycin and that demonstrate deoxycycline-induce luciferase activity when transiently transfected with pUHC13-3 are then tested for deoxycycline-inducible transcription factor expression by Northern and Western analysis as described below.

### Method 2. Conditional Expression of Transcription Factors Involved in Beta Cell Development and Maintenance Using AP1510-regulated Systems

Recently ARIAD Pharmaceuticals, Inc, has developed a inducible-gene expression system based on a bipartite transcription factor whose activity is regulated by a synthetic dimerization ligand (AP1510) (Belshaw et al., Proc. Natl. Acad. Sci. USA 93: 4604-4607 (1996); Ho et al., Nature 382: 822-826 (1996); Rivera et al., Nature Med. 2: 1028-1032 (1996)). The advantage of the ARIAD expression system is that basal expression of the target gene is very low, but gene expression can be induced to high levels by the addition of the dimerized ligand. This gene regulation system requires integration of two plasmids, pCEN-F3p65/Z15/neo and LH-Z₁₂-I-PL. The pCEN-P3p65/Z15/neo plasmid allows for the expression of two fusion proteins; one consisting of a DNA binding domain fused to FKBP12 and the other, a transcriptional activation domain fused to FKBP12 domains. The DNA-binding domain, called ZFHD1, is a composite of two transcription factors Zif268 and Oct-1 (Pomerantz et al., Science 267: 93-96 (1995)). The transactivation domain is derived from the C-terminal region of the NF-kB p65 protein (Schmitz and Baeurle, EMBO J. 10: 3805-3817 (1991)). The LH-Z₁₂-I-PL plasmid is the target plasmid which contains a minimal interleuken-2 gene promoter regulated by 12 binding sites for ZFHD1 (Rivera et al., Nature Med. 2: 1028-1032 (1996)). The target gene (i.e., Pdx-1 or BETA2) whose expression is to be regulated is cloned in a polylinker site downstream from the minimal interleuken-2 gene promoter. Upon the addition of AP1510, the two FKBP12 fusion proteins containing the DNA binding domain and the transactivation domain dimerize, thus activating the bipartite transcription factor. Activation of the bipartite transcription factor leads to induced expression of the target gene.

The first vector to be stably integrated into the HPDE6c7 cells is the pCEN-F3p65/Z15 plasmid. This plasmid has a neomycin resistance gene for selection of drug resistant clones. However, because HPDE cells are already neomycin resistant, the neomycin resistance gene cannot be used as a selectable marker. To overcome this, cotransfect the pCEN-F3p65/Z15 plasmid with the puromycin selection plasmid pPUR (Clontech, Palo Alto, Calif.). Then select for puromycin resistant HPDE clones and test these clones for AP1510-regulated reporter gene (LH-Z₁₂-I-S) expression. The LH-Z₁₂-I-S reporter gene contains the secreted alkaline phosphatase gene. Alkaline phosphatase activity in the media is measured using a kit from Tropix, Bedford, Mass.

### Characterization of Conditionally-Regulated HPDE Cells Expressing Transcription Factors Involved in Beta Cell Development and Maintenance

For all studies, proper controls are used. For experiments using recombinant adenovirus-mediated gene transduction, a recombinant adenovirus that expresses β-galactosidase is used as a control. Experiments using conditionally-regulated transcription factor gene expression have two controls: (1) conditionally-regulated cell lines that do not receive drug treatment (deoxycycline or AP1510); and, (2) conditionally-regulated cell lines that do not have the transcription factor cDNA inserted in the expression vector.

### A. Determination of Beta Cell-Specific mRNA Levels

Expression of beta cell-specific mRNA levels is determined by Northern analysis using various cDNAs as a hybridization probes as described in Section D, Specific Aim 1.

### B. Determination of Beta Cell-Specific Protein Expression

Expression of beta cell-specific proteins is determined by Western analysis using specific antiserum as described in Section D, Specific Aim 1.

### C. Insulin Secretion and Insulin Content

For static secretion studies, cells are plated in 12 well plates (22 mm diameter). After 1 to 7 days after expression of the transcription factors, cells are incubated twice for 30 min at 37° C. in glucose-free Krebs-Ringer buffer (KRB) and then incubated for 30 min with KRB containing various concentrations of glucose. Glucose concentrations examined range between 0.2 mM and 20 mM. Insulin secreted into the KRB is determined by a human insulin radioimmunoassay (RIA, Linco, St. Charles, Mo.). Insulin release is normalized to the concentration of either total protein or cellular DNA. Protein levels are determined by Lowry assay. Cellular DNA is measured by fluorometry. Insulin content is determined by RIA after acid-ethanol extraction (Santerre et al., Proc. Natl. Acad. Sci. USA 78: 4339-4343 (1981)).

Phasic insulin secretion in response to glucose is determined as follows. HPDE6c7 cells are subcultured for 2 days on glass cover slips. One to seven days after expression of the transcription factors, cells are perifused for 30 min with glucose-free KRB at a rate of 1 ml per min. The cells are then perifused for up to 1 hr with KRB containing increasing concentrations of glucose. In the event that the cells are poorly adhered to the cover slips, the cells are cultured in 12 well dishes and then perifused as a cell suspension. Insulin secretion is normalized to either protein or DNA concentration as described above.

In addition to glucose-induced insulin release, the effect that other secretagogues have on insulin release and the potentiation of glucose-induced insulin release is examined. These additional secretagogues include acetylcholine, GLP-1, leucine, arginine, and sulfonylureas. Testing the effect of these secretagogues on insulin release provides additional information on the functional state of the HPDE6c7 cells.

### D. Determining the Effect of Beta Cell-Specific Transcription Factors on HPDE Cell Proliferation

Our preliminary studies have demonstrated that Pdx-1 expression in HPDE6c7 cells restricts cell proliferation. Therefore, we are interested in determining the extent of growth arrest and phase in the cell cycle the various transcription factors are restricting proliferation.

The method to assess cell proliferation is to measure the incorporation of 5-bromo-2′-deoxyuridine (BrdU) into replicating DNA using the BrdU detection kit marketed by Amersham Life Science, Inc. Twenty-four to 48 hrs after expression of beta cell-specific transcription factors, cells are incubated in a culture media supplemented with BrdU and 5-fluoro-2′-deoxyuridine. The cells are then fixed and BrdU detected with an anti-BrdU monoclonal antibody. Detection of the antibody bound to BrdU is achieved by using a peroxidase-conjugated anti-mouse antiserum. Quantifying BrdU is achieved by measuring peroxidase activity using a microplate reader.

Cell cycle analysis is also performed to determine which phase of the cell cycle expression of the various beta cell-specific transcription factors arrests HPDE6c7 cell proliferation. HPDE6c7 cells are plated at a subconfluent density. After 24 to 48 hrs after transcription factor expression, cells are detached by mild trypsin-EDTA digestion, washed in PBS, and fixed in 80% ethanol. After fixation, the cells are stained in PBS containing 50 μg/ml propidium iodide and 0.01% RNase. The relative distribution of cells in G1, G2 and S phases of the cell cycle are then determined by flow cytometry on a Becton Dickinson FACS Vantage.

There are potential pitfalls to the above. The developmental program for generation of pancreatic beta cells is complex and requires the correct timing and levels of expression of a variety of transcription factors. Because of this, it is possible that expression of just one transcription factor may not be sufficient to drive HPDE cells towards a beta cell phenotype. Nonetheless, Pdx-1 seems to be the most reasonable transcription factor to test for the following reasons: (1) Pdx-1 is expressed early in the development of the pancreas (Ohlsson et al., EMBO J. 12: 4251-4259 (1993); Leonard et al., Mol. Endo. 7: 1275-1283 (1993); Miller et al., EMBO J. 13: 1145-1156 (1994)) and it is expressed at high levels in mature beta cells (Ohlsson et al., EMBO J. 12: 4251-4259 (1993); Guz et al., Development 121: 11-18 (1995)); (2) selective disruption of the pdx-1 gene decreases expression of other beta cell specific genes (Ahlgren et al., Genes & Development 12: 1763-1768 (1998)); (3) regeneration of beta cells from ductal cells coincides with increase Pdx-1 protein levels (Sharma et al., Diabetes 48: 507-513 (1999)); and, (4) Pdx-1 expression may work upstream of other transcription factors including Pax4 and Nkx6.1 (Edlund, Diabetes 47: 1817-1823 (1998)); Ahlgren et al., Genes & Development 12: 1763-1768 (1998)).

An additional concern is that Pdx-1 is thought to regulate the differentiation state of delta cells and is known to regulate somatostatin gene transcription (Leonard et al., Mol. Endo. 7: 1275-1283 (1993); Miller et al., EMBO J. 13: 1145-1156 (1994)). Therefore, expression of Pdx-1 in HPDE6c7 cells may cause the cells to differentiate into somatostatin-producing cells. In fact, somatostatin gene expression was shown to markedly increase when Pdx-1 was overexpressed in the Trm-6 cell line derived from human fetal islets (Itkin-Ansari et al. Diabetes 47 (Supplement 1): A252 (1998)). Interestingly, Nkx2.2 appears to be critical for insulin gene expression, but is not expressed in somatostatin producing cells (Sussel et al., Development 125: 2213-221 (1998)), suggesting that Nkx2.2 may play a role in defining whether a cell becomes a delta cell or a beta cell. Therefore, one approach that can be used to prevent HPDE6c7 cells from differentiating into somatostatin-producing cells is to co-express both Pdx-1 and Nkx2.2 in HPDE cells.

If expression of Pdx-1 is insufficient to differentiate HPDE cells into beta cells, the effect of other transcription factors including BETA2, Isl-1, Pax4, Pax6, Nkx6.1, or Nkx2.2 will be determined. The effect of these transcription factors on HPDE6c7 differentiation is tested alone and in combination with Pdx-1. Furthermore, we will examine whether the addition of mitogenic or differentiation agents along with expression of transcription factors will direct cells towards a beta cell phenotype. For example, when α-TC1 cells were transfected with the Pdx-1 gene, insulin and glucokinase gene expression was low unless betacellulin was added to the cell culture media (Watada et al., Diabetes 45: 1826-1831 (1996)).

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

We claim:

1. A human pancreatic ductal cell line immortalized with human papilloma virus genes E6 and E7 and which is capable of producing insulin, wherein the cells are gap junctional intercellular communication competent and are capable of expressing connexin43 gap junction protein upon induction by agents stimulating the production of cyclic AMP.

2. An immortalized human pancreatic ductal cell line capable of producing insulin and expressing connexin43 gap junction protein derived by differentiation from normal human pancreatic duct epithelium gap junctional intracellular communication incompetent cells transfected with human papilloma virus genes E6 and E7 and available from Michigan State University, East Lansing, Mich. or the Department of Laboratory Medicine and Pathobiology, University Health Network, Toronto, Ontario, Canada.

3. The cell line of claim 1, wherein the human papilloma virus genes E6 and E7 are provided by a retrovirus.

4. The cell line of claim 1, 2, or 3, wherein the cells are maintained in a medium comprising a three-dimensional matrix, which produces the connexin43 protein.

5. A pluripotent human pancreatic ductal cell line immortalized with human papilloma virus genes E6 and E7 and which is capable of producing insulin, wherein the cells are (i) contact inhibited in complete keratinocyte serum-free medium containing growth factors, hormones and bovine pituitary extract (KSFM), (ii) capable of forming tubular/ductal structures in a medium comprising a three-dimensional matrix, (iii) gap junction intercellular communication competent in keratinocyte basal medium (KBM), and (iv) capable of expressing connexin 32 and 43 genes in KBM comprising c-AMP elevating agents.

6. The cell line of claim 5, wherein the human papilloma virus genes E6 and E7 are provided by a plasmid or a recombinant virus.

7. The cell line of claim 5, wherein the cell line is HPDE6c7 deposited as ATCC ______.

8. The cell line of claim 5, wherein the cell line is maintained as the pluripotent stem cell line in KSFM.

9. The cell line of claim 5, wherein the cell line is maintained as a differentiated cell line in a medium selected from the group consisting of KBM, KBM with C-AMP elevating agents, and a medium comprising a three-dimensional matrix.

10. The cell line of claim 5 or 9, wherein the c-AMP elevating agents are selected from the group consisting of 3-isobutyl-1-methylxanthine, forskolin, and mixture thereof.

11. A method for screening a chemical agent for determining an affect on cells which comprises:
(a) providing a human pancreatic ductal cell line immortalized with human papilloma virus genes E6 and E7 and which is capable of producing insulin, wherein the cells are gap junctional communication competent and are capable of expressing connexin43 gap junction protein upon induction by agents stimulating the production of cyclic AMP; and
(b) exposing the cell line to the chemical agent to screen the effect of the chemical agent on the cell line.

12. The method of claim 11, wherein the immortalized human pancreatic ductal cell line is derived by differentiation from normal human pancreatic duct epithelium gap junctional intracellular communication incompetent cells transfected with human papilloma virus genes E6 and E7 and available from Michigan State University, East Lansing, Mich. or the Department of Laboratory Medicine and Pathobiology, University Health Network, Toronto, Ontario, Canada.

13. The method of claim 11, wherein the human papillomavirus genes E6 and E7 are provided by a retrovirus.

14. The method of claim 11, 12, or 13, wherein the cells are maintained in a medium comprising a three-dimensional matrix, which produces the connexin43 protein.

15. The method of claim 11 or 12, wherein the chemical agent is tested on the cell line for an ability to cause the cell line to become tumorigenic.

16. The method of claim 11, wherein the chemical agent is tested on the cell line for an ability to affect the production of insulin.

17. A method for differentiating cells which comprises:
(a) providing normal human pancreatic duct epithelium cells transfected with human papilloma virus genes E6 and E7, wherein the cells are gap junctional intracellular connection incompetent and are incapable of producing insulin and connexin43; and
(b) maintaining the cells of step (a) with a cyclic AMP elevating agent in basal medium, without hormones and growth factors, to produce the differentiated cells which are gap junctional intracellular connection competent and which produce connexin43 gap junction protein.

18. The method of claim 17, wherein the human papillomavirus E6 and E7 genes are provided by a retrovirus.

19. The method of claim 17, wherein an immortalized human pancreatic ductal cell line is derived by differentiation from normal human pancreatic duct epithelium gap junctional intracellular communication incompetent cells transfected with human papilloma virus genes E6 and E7 and available from Michigan State University, East Lansing, Mich.

20. The method of claim 17, 18, or 19, wherein the cells are maintained in a medium comprising a three-dimensional matrix, which produces the connexin43 protein.

21. A method for determining the ability of a chemical agent to affect differentiation of insulin-producing cells or tissues, which comprises:
(a) providing a pluripotent human pancreatic ductal cell line immortalized with human papilloma virus genes E6 and E7 and which is capable of producing insulin, wherein the cells are (i) contact inhibited in complete keratinocyte serum-free medium containing growth factors, hormones and bovine pituitary extract (KSFM), (ii) capable of forming tubular/ductal structures in a medium comprising a three-dimensional matrix, (iii) gap junction intercellular communication competent in keratinocyte basal medium (KBM), and (iv) capable of expressing connexin 32 and 43 genes in KBM comprising c-AMP elevating agents;
(b) exposing the cell line to the chemical agent in complete medium or basal medium with or without c-AMP elevating agents; and
(c) determining the effect of the chemical agent on differentiation.

22. The method claim 21, wherein the human papilloma virus genes E6 and E7 are provided by a plasmid or a recombinant virus.

23. The method of claim 21, wherein the cell line is HPDE6c7 deposited as ATCC ______.

24. The method of claim 21, wherein the c-AMP elevating agents are selected from the group consisting of 3-isobutyl-1-methylxanthine, forskolin, and mixture thereof.

25. A method for determining the ability of a chemical agent to affect production of insulin, which comprises:
(a) providing a pluripotent human pancreatic ductal cell line immortalized with human papilloma virus genes E6 and E7 and which is capable of producing insulin, wherein the cells are (i) contact inhibited in complete keratinocyte serum-free medium containing growth factors, hormones and bovine pituitary extract (KSFM), (ii) capable of forming tubular/ductal structures in a medium comprising a three-dimensional matrix, (iii) gap junction intercellular communication competent in keratinocyte basal medium (KBM), and (iv) capable of expressing connexin 32 and 43 genes in KBM comprising c-AMP elevating agents;
(b) exposing the cell line to the chemical agent in complete medium or basal medium with or without c-AMP elevating agents; and
(c) determining the effect of the chemical agent on production of insulin.

26. The method claim 25, wherein the human papilloma virus genes E6 and E7 are provided by a plasmid or a recombinant virus.

27. The method of claim 25, wherein the cell line is HPDE6c7 deposited as ATCC ______.

28. The method of claim 25, wherein the c-AMP elevating agents are selected from the group consisting of 3-isobutyl-1-methylxanthine, forskolin, and mixture thereof.

29. A method for determining the ability of a chemical agent to induce malignant proliferation of insulin-producing cells or tissues, which comprises:
(a) providing a pluripotent human pancreatic ductal cell line immortalized with human papilloma virus genes E6 and E7 and which is capable of producing insulin, wherein the cells are (i) contact inhibited in complete keratinocyte serum-free medium containing growth factors, hormones and bovine pituitary extract (KSFM), (ii) capable of forming tubular/ductal structures in a medium comprising a three-dimensional matrix, (iii) gap junction intercellular communication competent in keratinocyte basal medium (KBM), and (iv) capable of expressing connexin 32 and 43 genes in KBM comprising c-AMP elevating agents;
(b) exposing the cell line to the chemical agent in complete medium or basal medium with or without c-AMP elevating agents; and
(c) determining whether the cells undergo malignant proliferation.

30. The method claim 29, wherein the human papilloma virus genes E6 and E7 are provided by a plasmid or a recombinant virus.

31. The method of claim 29, wherein the cell line is HPDE6c7 deposited as ATCC ______.

32. The cell line of claim 29, wherein the c-AMP elevating agents are selected from the group consisting of 3-isobutyl-1-methylxanthine, forskolin, and mixture thereof.

33. A method for treating type-I diabetes in a mammal comprising:
(a) providing a therapeutically effective amount of a human pancreatic ductal cell line immortalized with human papilloma virus genes E6 and E7 and which is capable of producing insulin, wherein the cells are gap junctional intercellular communication competent and are capable of expressing connexin43 gap junction protein upon induction by agents stimulating the production of cyclic AMP, positioned in a means for producing an artificial pancreas; and
(b) implanting the artificial pancreas in the mammal wherein the artificial pancreas produces insulin.

34. The method of claim 33, wherein the immortalized human pancreatic ductal cell line is derived by differentiation from normal human pancreatic duct epithelium gap junctional intracellular communication incompetent cells transfected with human papilloma virus genes E6 and E7 and available from Michigan State University, East Lansing, Mich.

35. The method of claim 33, wherein the artificial pancreas comprises the immortalized cell line positioned within a selectively permeable device which is connected to the vasculature of the mammal.

36. A human pancreatic ductal epithelial cell line wherein the cells of the cell line are immortalized with an agent selected from the group consisting of human papilloma virus (HPV) genes E6 and E7, SV40 T antigen, Rous sarcoma virus, one or more oncogenes selected from the group consisting of ras, scr, and neu, and a chemical mutagen selected from the group consisting of N-methyl-N-nitro-N-nitrosoguanidine (MNNG), methyl methane sulfonate(MMS), nitrosourea (NMU), dimethylbenz[a]anthracine (DBMA), 4-nitroquinoline-N-oxide (NQO), and nickel(II) and which is capable of producing insulin, wherein the cells are gap junctional intercellular communication competent and are capable of expressing connexin43 gap junction protein upon induction by agents stimulating the production of cyclic AMP.

37. A method for making an immortalized human pancreatic ductal epithelial cell line which is capable of producing insulin, wherein the cells are gap junctional intercellular communication competent and are capable of expressing connexin43 gap junction protein upon induction by agents stimulating the production of cyclic AMP, comprising:
(a) isolating ductal tissue from human pancreatic tissue;
(b) incubating the ductal tissue in a cell culture to form a monolayer of cells growing from the ductal tissue;
(c) treating the monolayer of cells with an agent selected from the group consisting of human papilloma virus (HPV) genes E6 and E7, SV40 T antigen, Rous sarcoma virus, one or more oncogenes selected from the group consisting of ras, scr, and neu, and a chemical mutagen selected from the group consisting of N-methyl-N-nitro-N-nitrosoguanidine (MNNG), methyl methane sulfonate(MMS), nitrosourea (NMU), dimethylbenz[a]anthracine (DBMA), 4-nitroquinoline-N-oxide (NQO), and nickel(II) for a time sufficient to immortalize the cells; and
(d) growing the immortalize cells for a time sufficient to allow the cells that are not immortalized to die to produce the immortalized cell line, wherein the immortalized cell line is capable of producing insulin, and wherein the immortalized cells of the cell line are gap junctional intercellular communication competent and are capable of expressing connexin43 gap junction protein upon induction by agents stimulating the production of cyclic AMP.


SEQUENCE LISTING (10 sequences)

SEQ ID NO: 1 - DNA, 19 bp, Artificial Sequence
  Human connexin45 5′ PCR primer
ggagcacgctgaagcagac

SEQ ID NO: 2 - DNA, 18 bp, Artificial Sequence
  human connexin45 3′ PCR primer
cgggtggacttggagcca

SEQ ID NO: 3 - DNA, 21 bp, Artificial Sequence
  Human beta-actin 5′ PCR primer
cggcatcgtcaccaactggga

SEQ ID NO: 4 - DNA, 19 bp, Artificial Sequence
  Human beta-actin 3′ PCR primer
cgtagatgggcagtgtggg

SEQ ID NO: 5 - DNA, 21 bp, Artificial Sequence
  Pdx-1 5′ PCR primer
gataagaaacgtagtagcggg

SEQ ID NO: 6 - DNA, 21 bp, Artificial Sequence
  Pdx-1 3′ PCR primer
cgacgtggcgcgacgctggag

SEQ ID NO: 7 - DNA, 23 bp, Artificial Sequence
  human connexin36 5′ PCR primer
acgccgctactctacagtcttcc

SEQ ID NO: 8 - DNA, 24 bp, Artificial Sequence
  human connexin36 3′ PCR primer
gatgccttcctgccttctgagctt

SEQ ID NO: 9 - DNA, 20 bp, Artificial Sequence
  human GAPDH 5′ PCR primer
gttcgacagtcagccgcatc

SEQ ID NO: 10 - DNA, 22 bp, Artificial Sequence
  human GAPDH 3′ PCR primer
gtgggtgtcggctgttgaagtc